SEQUENCE LISTING (25 sequences)

SEQ ID NO: 1 - RNA, 19 nt, artificial
  Human NR2F2 siRNA
gccgucucaagaagugcuu

SEQ ID NO: 2 - RNA, 19 nt, artificial
  Human NR2F2 siRNA
cauugagacacugaucaga

SEQ ID NO: 3 - RNA, 19 nt, artificial
  Human NR2F2 siRNA
gcaagcauuacggugucuu

SEQ ID NO: 4 - RNA, 19 nt, artificial
  Human NR2F2 siRNA
ccccuagcaugaacuugug

SEQ ID NO: 5 - DNA, 1804 bp, Homo sapiens
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 6 - DNA, 2217 bp, Mus musculus
gcgccgatggaacgcgggtgtcaggccggccgcagcgcggggccggcggcgagcgccagggcgaggccgaggctcgggcccaggcgcaggccgaggccggccgcgcgagcgctcggcggggagacgatccagggaaggccgcgggtcgcactctccactcagctctatcgcctggacctctgcgattacggccgggcgcgcgcggcgtgcgggactccgggtctccgacgcgcgctcccgccgcccctcccccctcgccgcgtaacttgcggccaaagtttccccccgggctcgggggcgcccgcgcgcgctcggatggtgagccactaagttggcctgggcggcggggccgggccatggcccccgcgacgctaccgggtccccaggactccggaccacgggacctgggcgccccagactcgcgcctctagcgcgcccccgtcgaccgcgggcacgcgtgggaaagttggcctggaaccggcccgaccagttcctgcctggcgcgcggaccggccgcaggaagttgccgcaaaacttttttcaggggggtgtgcgaccggagccccccgagagcgcgggctgcatgcgcccggggtagccgggtccctctcgggtcgccaggcgtgcccagaggggacggactcgtcccggggcgtaccggccccgctgtctccggggctatggccatggtgaccggtggctggggcgaccccggaggcgacacgaacggcgtggacaaggctggtgggagctacccacgcgcgaccgaggacgattcggcgtcacctcccggggcgaccagcgacgcggagccgggcgacgaggagcgtccggggttgcaggtggactgcgtggtgtgcggggacaagtccagtggaaagcattacggcgtgttcacctgcgagggctgcaagagtttcttcaagcgcagcatccgccgcaatctcagctacacctgccggtccaaccgtgactgtcagattgatcagcaccaccggaaccagtgtcagtactgtcggctcaagaagtgcttccgggtgggcatgcgcaaggaggccgtgcagcgaggccgcatcccgcatgcgctccccggtccagcggcctgcagtcccccgggcgcgacgggcgtcgaacctttcacggggccgccagtgtccgagctgattgcgcagctgctgcgtgctgagccctaccccgcggccggacgctttggtggcggcggcgctgtactgggcatcgacaacgtgtgcgagttggcggcacgcctgctgttcagcacggtcgagtgggcccgccacgcgcccttcttccccgagctgccggccgccgaccaggtggcgctgctgcggctcagctggagtgagctcttcgtgctgaacgcggcgcaggcggcgctgccgctgcatacggcaccgctgctggccgccgcggggttgcatgccgcgcccatggcagccgagcgggccgtggccttcatggaccaggtgcgtgccttccaggagcaggtggacaagctgggccgcctgcaggtggatgctgcggagtacggctgcctcaaggccatcgcgctcttcacgcctgatgcctgtggcctttctgacccagcccatgtggagagcctgcaggagaaggcacaggtggccctcaccgagtatgtgcgtgcccagtacccatcgcagccccagcgctttgggcgtctgctgctgcggctgccagccctgcgtgctgtgcccgcatccctcatctcccagctcttcttcatgcgcctggtgggcaagacacccatcgagaccctcatccgggacatgcttctgtcagggagcacctttaactggccctatggctcgggctagtgatagtcaccttccaggacatacatggaaactggggccttgtggggaccctggggatcagggccccagcttctcttttgagactgatttctttttttaaagactgtgaaatgtttgttttgttttattttttaaataatcatgaaaccaaaaagatttggatctcccaggccttgtcctggcagaccttcaacagtctggagccagcatgctgatgcctctggtgtcatgggtatctggaaaggccactgcagctaggcaggagtactatgggccaggaggatcccctggatacatggtccacggagggcaccatgggatgatgaaaacctggccaataataaaggtattcccttacttggtc

SEQ ID NO: 7 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 8 - protein, 390 aa, Mus musculus
MAMVTGGWGDPGGDTNGVDKAGGSYPRATEDDSASPPGATSDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHALPGPAACSPPGATGVEPFTGPPVSELIAQLLRAEPYPAAGRFGGGGAVLGIDNVCELAARLLFSTVEWARHAPFFPELPAADQVALLRLSWSELFVLNAAQAALPLHTAPLLAAAGLHAAPMAAERAVAFMDQVRAFQEQVDKLGRLQVDAAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSG

SEQ ID NO: 9 - RNA, 19 nt, artificial
  human siRNA
gccgucucaagaagugcuu

SEQ ID NO: 10 - RNA, 19 nt, artificial
  human siRNA
cauugagacacugaucaga

SEQ ID NO: 11 - RNA, 19 nt, artificial
  human siRNA
gcaagcauuacggugucuu

SEQ ID NO: 12 - RNA, 19 nt, artificial
  human siRNA
ccccuagcaugaacuugug

SEQ ID NO: 13 - DNA, 64 bp, artificial
  mus shNR2F6.1
gatccgcattacggcgtgttcaccttcaagagaggtgaacacgccgtaatgcttttttctagag

SEQ ID NO: 14 - DNA, 65 bp, artificial
  mus shNR2F6.2
gatccgcaaccgtgactgtcagattaagttctctaatctgacagtcacggttgttttttctagag

SEQ ID NO: 15 - DNA, 65 bp, artificial
  mus shNR2F6.3
gatccgtgtccgagctgattgcgcattcaagagatgcgcaatcagctcggacattttttctagag

SEQ ID NO: 16 - DNA, 64 bp, artificial
  human shNR2F6.1
gatccgcattacggtgtcttcaccttcaagagaggtgaagacaccgtaatgcttttttctagag

SEQ ID NO: 17 - DNA, 65 bp, artificial
  human shNR2F6.2
gatccgcctctggacacgtaacctattcaagagataggttacgtgtccagaggttttttctagag

SEQ ID NO: 18 - DNA, 21 bp, artificial
  Human NR2F6 Forward Primer
tctcccagctgttcttcatgc

SEQ ID NO: 19 - DNA, 20 bp, artificial
  Human NR2F6 Reverse Primer
ccagttgaaggtactccccg

SEQ ID NO: 20 - DNA, 20 bp, artificial
  Human GAPDH Forward Primer
ggcctccaaggagtaagacc

SEQ ID NO: 21 - DNA, 20 bp, artificial
  Human GAPDH Reverse Primer
aggggtctacatggcaactg

SEQ ID NO: 22 - DNA, 19 bp, artificial
  3′ end Mus NR2F6 Forward Primer
cctggcagaccttcaacag

SEQ ID NO: 23 - DNA, 19 bp, artificial
  3′ end Mus NR2F6 Reverse Primer
gatcctcctggcccatagt

SEQ ID NO: 24 - DNA, 20 bp, artificial
  3′ end Mus L32 Forward Primer
gccatcagagtcaccaatcc

SEQ ID NO: 25 - DNA, 20 bp, artificial
  3′ end Mus L32 Reverse Primer
aaacatgcacacaagccatc

(19) United States
(12) Patent Application Publication
(10) Pub. No.: US 2015/0299712 A1
(43) Pub. Date: Oct. 22, 2015

(54) Modulation of Hematopoietic Stem Cell Differentiation

(71) Applicants: Christine Victoria Ichim, Spring Valley, CA (US); Tom Ichim, San Diego, CA (US)

(72) Inventors: Christine Victoria Ichim, Spring Valley, CA (US); Tom Ichim, San Diego, CA (US)

(21) Appl. No.: 14/595,078

(22) Filed: Jan. 12, 2015

Related U.S. Application Data
(63) Continuation-in-part of application No. 13/652,395, filed on Oct. 15, 2012, now Pat. No. 9,091,696.
Application No. 13/652,395 is a continuation-in-part of application No. 12/619,290, filed on Nov. 16, 2009, now abandoned.
(60) Provisional application No. 61/114,764, filed on Nov. 14, 2008.

(51) Int. Cl. C12N 15/113

(52) U.S. Cl. CPC C12N 15/1138; C12N 2310/11; C12N 2310/531; C12N 2320/30

(57) ABSTRACT

The invention provides means of manipulating hematopoietic stem cell differentiation by modulation of levels of NR2F6 (EAR2). Provided are compositions of matter, protocols and methods of use by which inhibiting expression of NR2F6 or activity thereof promotes differentiation of selective hematopoietic lineages, or conversely overexpression of NR2F6 or activity thereof inhibits differentiation. In one embodiment inhibition of differentiation is performed in other cell lineages besides hematopoietic.

### CROSS-REFERENCE TO RELATED APPLICATIONS

This application claims priority to and is a continuation-in-part to pending Non-Provisional U.S. application Ser. No. 13/652,395 filed Oct. 15, 2012, which claims priority to Non-Provisional U.S. application Ser. No. 12/619,290, filed Nov. 16, 2009, which claims the benefit under 35 USC §119(e) of U.S. provisional application No. 61/114764 filed Nov. 14, 2008, each of which is hereby expressly incorporated by reference in their entirety.

### FIELD OF THE INVENTION

The invention pertains to the field of stem cell differentiation, more specifically the invention relates to the field of inducing stem cell differentiation through the inhibition of NR2F6 and conversely inhibition of differentiation through up regulation of NR2F6 expression. The invention further relates to modulation of hematopoietic stem cell differentiation by manipulation of levels of NR2F6.

### BACKGROUND

Control of stem cell differentiation would allow for expansion of primitive stem cell or progenitor cell populations, thus allowing for various cellular therapeutics that are currently limited by inability to expand sufficient cells. For example, it is known that c-kit positive cardiac progenitor cells reside in adult hearts. Studies have shown that ex vivo expanded and subsequently replanted cardiac progenitor cells possess therapeutic benefits. Unfortunately expansion of these cells ex vivo is limited by spontaneous differentiation of the cardiac progenitors during culture. Additionally, ex vivo expansion of hematopoietic stem cells is limited by spontaneous differentiation. The ability to expand cells such as cord blood hematopoietic stem cells without differentiation would allow for larger populations of cells that can be allogeneically transplanted with lower matching requirements as compared to bone marrow derived hematopoietic stem cells. In the art there is a need for inducing differentiation of hematopoietic stem cells. For example, chemo- and radiation therapies cause dramatic reductions in blood cell populations in cancer patients. At least 500,000 cancer patients undergo chemotherapy and radiation therapy in the US and Europe each year and another 200,000 in Japan. Bone marrow transplantation therapy of value in aplastic anemia, primary immunodeficiency and acute leukemia (following total body irradiation) is becoming more widely practiced by the medical community. At least 15,000 Americans have bone marrow transplants each year. Other diseases can cause a reduction in entire or selected blood cell lineages. Examples of these conditions include anemia (including macrocytic and aplastic anemia); thrombocytopenia; hypoplasia; immune (autoimmune) thrombocytopenic purpura (ITP); and HIV induced ITP. The current invention describes a modulatory switch found in stem cells generally and also in hematopoietic stem cells.

In the area of hematopoietic stem cell (HSC) self-renewal roles for several proteins have been identified. These include pathways involved in embryonic development (Wnt/β-catenin [6], Notch/Delta-like [7], BMP/SMADs [8]), the hox genes and their partners (Cdx [9], Hoxa9 [10], Hoxa10 [11], Hoxb4 [12], Meis [9], Pbx [9]), and polycomb/trithorax group genes (Bmi1 [13, 14], Mll [15]). In addition, a number of transcription factors involved in blood cell differentiation have also been shown to be necessary for self-renewal (Gata-2 [16], Gfi1 [17], JunB [18], Pu.1 [19], Myb [20], Cbp [21], Myc [22], and Zfx [23]). How these diverse pathways are integrated in vivo is not understood; it has been postulated that epigenetic modifications such as chromatin and histone methylation and acetylation play a key role [24], and that the switch between HSC self-renewal and differentiation is regulated by competition between transcription factor complexes, akin to the interplay among Gata-1, c/ebpa, and Pu.1 that mediates the myeloid/erythroid lineage decision [25, 26]. However, proteins that selectively modulate differentiation versus self-renewal, and that possess specificity towards certain lineages are not known.

The invention provides means of inhibiting stem cell differentiation by induction of NR2F6 upregulation. Conversely the invention provides means of inducing stem cell differentiation by inhibiting activity of NR2F6.

### SUMMARY OF THE INVENTION

The invention provides means of modulating differentiation of stem cells. Specifically, the invention teaches that overexpression of NR2F6 induces blockade of differentiation in stem cells, whereas suppression of NR2F6 augments stem cell differentiation. One specific aspect of the invention provides the induction of T cell differentiation from a HSC or progeny thereof by reducing expression of NR2F6, or conversely blocking T cell differentiation by overexpressing NR2F6.

Several aspects of the invention are presented below in claim format:

1. In one embodiment, the invention is a method of modulating differentiation of a stem cell comprising the steps of: a) selecting a stem cell; b) treating said stem cell with an agent that modulates activity of NR2F6.

2. In another embodiment, the invention is the method of claim 1, wherein said differentiation of said stem cells into a cell of increased maturity is achieved through inhibition of NR2F6 activity.

3. In another embodiment, the invention is the method of claim 2, wherein inhibition of differentiation of said stem cell is achieved through induction of increased NR2F6 activity.

4. In another embodiment, the invention is the method of claim 1 wherein said stem cell is a progenitor cell.

5. In another embodiment, the invention is the method of claim 1, wherein said stem cell is selected from a group comprising of embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

6. In another embodiment, the invention is the method of claim 5, wherein said embryonic stem cells are totipotent and express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

7. In another embodiment, the invention is the method of claim 5, wherein said cord blood stem cells are multipotent and capable of differentiating into endothelial, smooth muscle, and neuronal cells.

8. In another embodiment, the invention is the method of claim 7, wherein said cord blood stem cells are identified based on expression of one or more antigens selected from a group comprising: SSEA-3, SSEA-4, CD9, CD34, c-kit, OCT-4, Nanog, and CXCR-4

9. In another embodiment, the invention is the method of claim 8, wherein said cord blood stem cells do not express one or more markers selected from a group comprising of: CD3, CD34, CD45, and CD11b.

10. In another embodiment, the invention is the method of claim 13, wherein said placental stem cells are isolated from the placental structure.

11. In another embodiment, the invention is the method of claim 10, wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

12. In another embodiment, the invention is the method of claim 5, wherein said bone marrow stem cells comprise of bone marrow mononuclear cells.

13. In another embodiment, the invention is the method of claim 12, wherein said bone marrow stem cells are selected based on the ability to differentiate into one or more of the following cell types: endothelial cells, smooth muscle cells, and neuronal cells.

14. In another embodiment, the invention is the method of claim 13, wherein said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

15. In another embodiment, the invention is the method of claim 10, wherein said placental stem cells are isolated from the Wharton's Jelly.

16. In another embodiment, the invention is the method of claim 10, wherein said placental stem cells are mesenchymal in morphology.

17. In another embodiment, the invention is the method of claim 16, wherein said placental cell expresses one or more cytokines associated with a regenerative activities.

18. In another embodiment, the invention is the method of claim 17, wherein said cytokines associated with regenerative activity as selected from a group comprising of CS-6, IL-6, IL-8, SDF-1, CXCL5, VEGF, CXCL6, COL4A4, MMP13, CYP7B1, ADAMDEC1, SLC6A1, CXCL1, PF4V1, CXCL3, CH25H, SFRP2, MMP1, DARC, HCK, bFGF, ERC2, CLIC6, and BCL8 in a 75% confluent culture.

19. In another embodiment, the invention is the method of claim 18, wherein said placental stem cells exhibit expression of about 0.0-2,200.0 pg/ml of each of the one or more cytokines in a 75% confluent culture.

20. In another embodiment, the invention is the method of claim 18, wherein said placental cell exhibits expression of between 200.0-1900.0 pg/ml of IL-6 in a 75% confluent culture.

21. In another embodiment, the invention is the method of claim 18, wherein, said cell expresses between 350.0-2,000.0 pg/ml of bFGF in a 75% confluent culture.

22. In another embodiment, the invention is the method of claim 18, wherein said cell expresses between 500.0-2,500.0 pg/ml of VEGF in a 75% confluent culture.

23. In another embodiment, the invention is the method of claim 18, wherein said cell expresses between 140.0-1,500.0 pg/ml of SDF-1 in a 75% confluent culture.

24. In another embodiment, the invention is the method of claim 1, wherein said cell is an immortalized cell with regenerative activity.

25. In another embodiment, the invention is the method of claim 24, wherein said cell population is immortalized by means selected from a group comprising of; a) transfection with an oncogene; b) transfection telomerase; and c) transfection with a combination of an oncogene and telomerase.

26. In another embodiment, the invention is the method of claim 25, wherein said cell population is immortalized by means of transfection with an oncogene selected from a group of oncogenes comprising of: a) abl; b) Af4/hrx; c) akt-2; d) alk; e) alk/npm; f) aml1; g) aml1/mtg8; h) bc1-2, 3, 6; i) bcr/abl; j) c-myc; k) dbl; l) dek/can; m) E2A/pbx1; n) egfr; o) enl/hrx; p) erg/TLS; q) erbB; r) erbB-2; s) ets-1; t) ews/fli-1; u) fms; v) fos; w) fps; x) gli; y) gsp; z) gsp; aa) HER2/new; ab)hox11; ac) hst; ad) IL-3; ae) int-2; af) jun; ag) kit; ah) KS3; ai) K-sam; aj)Lbc; ak) lck; al) Imol, Imo-2; am) L-myc; an) lyl-1; ao) lyt-10; ap)lyt-10/C alpha 1; aq) mas; ar) mdm-2; as) mll; at) mos; au) mtg8/aml1; av) myb; aw) MYH11; ax) new; ay) N-myc; az) ost; ba) pax-5; bb) pbx1/E2a; bc) pim-1; bd) PRAD-1; be) raf; bf) RAR/PML; bg) Ras H, K, N; bh) rel/nrg; bi) ret; bj) rhom1, rhom2; bk) ros; bl) ski; bm) sis; bn) set/can; bo) src; bp) Tal1, tal2; bq) tan-1; br) Tiam1; bs) TSC2; and bt) trk.

26. In another embodiment, the invention is the method of claim 5, wherein said adipose stem cell expresses markers selected from a group comprising of: CD13, CD29, CD44, CD63, CD73, CD90, CD166, Aldehyde dehydrogenase (ALDH), and ABCG2.

27. In another embodiment, the invention is the method of claim 26, wherein said adipose tissue derived stem cells are a population of purified mononuclear cells extracted from adipose tissue capable of proliferating in culture for more than 1 month.

28. In another embodiment, the invention is the method of claim 5, wherein said side population cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

29. In another embodiment, the invention is the method of claim 1 wherein NR2F6 activity is inhibited by suppression of expression of the NR2F6 gene, said suppression of expression achieved by treatment of stem cells with an oligonucleotide capable of inhibiting expression of NR2F6 gene.

30. In another embodiment, the invention is the method of claim 29, wherein suppression of NR2F6 gene expression is achieved by treatment of said stem cell with a oligonucleotide comprising of a short-interfering ribonucleic acid (siRNA) molecule.

31. In another embodiment, the invention is the method of claim 29, wherein suppression of NR2F6 gene expression is achieved by treatment of said stem cell with a synthetic oligonucleotide comprising of a short-hairpin ribonucleic acid (shRNA) molecule.

32. In another embodiment, the invention is the method of claim 29, wherein suppression of NR2F6 gene expression is achieved by treatment of said stem cell with an oligonucleotide consists of an antisense ribonucleic acid molecule.

33. In another embodiment, the invention is the method of claim 29 wherein the effective portion of the oligonucleotide consists of SEQ ID NO: 9, 10, 11 or 12

34. In another embodiment, the invention is the method of claim 29 wherein the effective portion of the oligonucleotide consists of SEQ ID NO: 16 or 17

35. In another embodiment, the invention is the method of claim 29 wherein the effective portion of the oligonucleotide consists of SEQ ID NO: 13, 14 or 15

36. In another embodiment, the invention is the method of claim 29 comprising administering to said stem cell an oligonucleotide complementary to a nuclear receptor having a mRNA sequence of at least 70% sequence identity to the mRNA sequence of SEQ ID NO: 5, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that is selected based on its ability to inhibits the expression of the nuclear receptor NR2F6 by causing degradation of a ribonucleic acid encoding nuclear receptor NR2F6.

37. In another embodiment, the invention is the method of claim 1, wherein augmentation of NR2F6 expression is performed through transfection with a construct constitutively expressing said NR2F6 gene.

38. In another embodiment, the invention is the method for enhancing hematopoietic differentiation of a mammalian stem cell comprising, transfecting said stem cells with an oligonucleotide capable of suppressing expression of NR2F6.

39. In another embodiment, the invention is the method of claim 38, wherein said oligonucleotide possesses at least 75% homology with SEQ IDs NO: 9, 10, 11 or 12

40. In another embodiment, the invention is the method of claim 38, wherein said oligonucleotide possesses at least 75% homology with SEQ IDs NO: 16 or 17

41. In another embodiment, the invention is the method of claim 38, wherein said oligonucleotide possesses at least 75% homology with SEQ IDs NO: 1, 2, 3 or 4

42. In another embodiment, the invention is the method of claim 38 comprising administering to said stem cell an oligonucleotide complementary to a nuclear receptor having a mRNA sequence of at least 70% sequence identity to the mRNA sequence of SEQ ID NO: 5, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that is selected based on its ability to inhibits the expression of the nuclear receptor NR2F6 by causing degradation of a ribonucleic acid encoding nuclear receptor NR2F6.

43. In another embodiment, the invention is the method of claim 38, wherein the stem cell is a hematopoietic stem cell.

44. In another embodiment, the invention is the method of claim 38, wherein the cell is a CD34+ cell.

45. In another embodiment, the invention is the method of claim 38, wherein the cell is autologous.

46. In another embodiment, the invention is the method of claim 38, wherein the cell is obtained from a human.

47. In another embodiment, the invention is the method of claim 46, wherein the human is suffering from, or is susceptible to, decreased blood cell levels.

48. In another embodiment, the invention is the method of claim 47, wherein the decreased blood cell levels are caused by chemotherapy, radiation therapy, bone marrow transplantation therapy or congenital anemia.

49. In another embodiment, the invention is the method of claim 38, wherein the exogenous nucleic acid is a retroviral vector.

50. In another embodiment, the invention is a method of treating a mammal in need of improved hematopoietic capability, comprising the steps of: (a) removing hematopoietic stem cells from the mammal; (b) transfecting said stem cells with an oligonucleotide capable of suppressing expression of NR2F6; (c) culturing said transfected stem cells to form an expanded population of stem cells; and (d) returning said expanded cells to the mammal, whereby hematopoietic capability is improved.

51. In another embodiment, the invention is the method of claim 50 wherein one strand of said oligonucleotide possesses at least 75% homology with SEQ IDs NO: 1, 2, 3 or 4

52. In another embodiment, the invention is the method of claim 50, wherein one strand of said oligonucleotide possesses at least 75% homology with SEQ IDs NO: 9, 10, 11 or 12

53. In another embodiment, the invention is the method of claim 50, wherein one strand of said oligonucleotide possesses at least 75% homology with SEQ IDs NO: 16 or 17

54. In another embodiment, the invention is the method of claim 50 comprising administering to said stem cell an oligonucleotide complementary to a nuclear receptor having a mRNA sequence of at least 75% sequence identity to the mRNA sequence of SEQ ID NO: 5, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that is selected based on its ability to inhibits the expression of the nuclear receptor NR2F6 by causing degradation of a ribonucleic acid encoding nuclear receptor NR2F6.

55. In another embodiment, the invention is the method of claim 50 wherein said oligonucleotide is delivered in the form of an siRNA, an shRNA or an antisense oligonucleotide.

56. In another embodiment, the invention is the method of claim 50, wherein the mammal is a human.

### BRIEF DESCRIPTION OF THE DRAWINGS

FIG. 1 shows that bone marrow cells that over-express NR2F6 do not contribute to peripheral blood T-cells in a chimerical bone marrow transplant model.

FIG. 2 shows that NR2F6 over-expressing bone marrow is at a competitive disadvantage to normal bone marrow with respect to survival and proliferation in the thymus of chimerical bone marrow transplant recipients, shown here in representative animals.

FIG. 3 shows that NR2F6 over-expressing bone marrow is at a competitive disadvantage to normal bone marrow with respect to survival and proliferation in the thymus of chimerical bone marrow transplant recipients, shown here as a summary of two independent experiments.

FIG. 4 shows a reduction in the size of the thymus in animals that had been transplanted with bone marrow that over-expresses NR2F6 in contrast to control bone marrow transplant recipients.

FIG. 5 shows a reduction in the cellularity of the thymic cortex in bone marrow transplant recipients that received bone marrow that over-expressed NR2F6.

FIG. 6 shows with better resolution a reduction in the cellularity of the thymic cortex in bone marrow transplant recipients that received bone marrow that over-expressed NR2F6.

FIG. 7 shows cell death in the thymic medulla in bone marrow transplant recipients that received bone marrow that over-expressed NR2F6. Evident here are the phagocytic cells with remnants of cellular debris in their cytoplam.

FIG. 8 show that NR2F6 is highly expressed in both long and short term haematopoietic stem cells and that expression of NR2F6 in bone marrow hierarchy is differentially expressed

FIG. 9 show a model whereby NR2F6 is a gatekeeper of lineage selection

FIG. 10 shows that over expression of NR2F6 in bone marrow cells inhibits progenitor cell colony formation in methylcellulose along the myeloid and erythroid lineages

FIG. 11 shows that over expression of NR2F6 in bone marrow cells inhibits differentiation of bone marrow cells evidenced by generation of smaller colonies in NR2F6 transduced cultures

FIG. 12 shows that over expression of NR2F6 in bone marrow cells inhibits differentiation of progenitor cells in suspension culture in to granulocytes. Granulocytes were assessed in cultures by flow cytometry for expression of the surface antigens CD11b and Gr-1.

FIG. 13 shows that modulating the expression of NR2F6 in a chimerical bone marrow mouse model (30% NR2F6 transduced, 70% untransduced cells) causes a block in the ability of hematopoietic stem cells to differentiate into hematopoietic progenitor cells. Specifically, the megakaryocyte-erythroid progenitor cell (MEP) while decreasing the size of the common myeloid progenitor cell population as well as the granulocyte-monocyte progenitor (GMP) cell population.

FIG. 14 shows that modulating the expression of NR2F6 in a chimerical bone marrow mouse model (30% NR2F6 transduced, 70% untransduced cells) increases hematopoietic stem cell differentiation into the common lymphoid progenitor cells.

FIG. 15 shows that mice that over expression of NR2F6 in their bone marrow cells have skewed production of immature blood cells in the bone marrow. At 5 weeks post transplantation we observed more c-kit+ cells in the bone marrow, indicative of an increase in immature cells. While at 12 weeks post transplant we observed increased production of B220+ cells (immature B-cells) and decreased production of CD11b+/GR1+ cells (granulocytes)

FIG. 16 shows that mice that over expression of NR2F6 in their bone marrow cells have a block at the pro-erythroblast stage of blood cell development both in the bone marrow and in the spleen of animals that have excessive expression of NR2F6

FIG. 17 shows serial replating of bone marrow from mice that over express NR2F6 in their bone marrow. Bone marrow was grown in methylcellulose medium containing cytokines that promoted multi lineage differentiation. We observe a decrease in the ability of the cells to differentiate, evidenced by fewer primary colonies, but observed an increase in the self-renewal of the bone marrow cells evidenced by ability to serially replate

FIG. 18 shows that NR2F6 impairs the formation of mature red blood cells in animals that over-express NR2F6 in their bone marrow. This is demonstrated by a reduction in radioprotection, survival following a dose of lethal radiation, in animals that received bone marrow that over-expressed NR2F6.

FIG. 19 shows aberrant gene expression in key genes that govern erythropoiesis in the pro-erythroblast population of animals that over-express NR2F6 and vector control animals

FIG. 20 shows aberrant gene expression in key genes that govern hematopoiesis in the KSL population of bone marrow stem and progenitor cells in animals that over-express NR2F6 and vector control animals

FIG. 21 shows quantification of NR2F6 (EAR-2) protein levels, determined by immunoblot and quantified using densitometry, in human 32Dc13 undifferentiated hematopoietic cells that were treated with NR2F6 shRNA or a hairpin control.

FIG. 22 shows that shRNA to NR2F6 rapidly induces differentiation of bone marrow stem cells ex vivo. Here we show a reduction in the number of KSL bone marrow cells remaining in ex vivo cultures.

FIG. 23 shows that shRNA to NR2F6 rapidly induces differentiation of bone marrow stem cells ex vivo. Here we show a reduction in the number of immature bone marrow cells that are devoid of lineage antigens.

FIG. 24 shows that shRNA to NR2F6 rapidly induces differentiation of bone marrow stem cells ex vivo. Here we show a reduction in the number of immature bone marrow cells that are devoid of lineage antigens.

FIG. 25 shows that shRNA to NR2F6 rapidly induces differentiation of bone marrow stem cells ex vivo. Here we show a cytospin preparation that shows morphologically a reduction in the number of immature bone marrow cells. Samples that were treated with NR2F6 shRNA rapidly differentiated in to granulocytic cells

FIG. 26 shows that shRNA to NR2F6 rapidly induces differentiation of bone marrow stem cells ex vivo. Here we show flow cytometry dot plots that shows that samples that were treated with NR2F6 shRNA rapidly differentiated in to granulocytic cells

FIG. 27 shows that shRNA to NR2F6 rapidly induces differentiation of bone marrow stem cells. Here we show that shRNA to NR2F6 inhibits the formation of secondary bone marrow colonies, hence demonstrating a reduction in the self-renewal ability of the cultures due to differentiation.

### DETAILED DESCRIPTION OF THE INTERVENTION

For the purposes of advancing and clarifying the principles of the invention disclosed herein, reference will be made to certain embodiments and specific language will be used to describe said embodiments. It will nevertheless be understood and made clear that no limitation of the scope of the invention is thereby intended. The alterations, further modifications and applications of the principles of the invention as described herein serve only as specific embodiment, however one skilled in the art to which the invention relates will understand that the following are indeed only specific embodiments for illustrative purposes, and will derive similar types of applications upon reading and understanding this disclosure.

To allow for the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The invention provides means and methods of modulating differentiation of a stem cell comprising the steps of: a) selecting a stem cell; b) treating said stem cell with an agent that modulates activity of NR2F6. In situations where said differentiation of said stem cells into a cell of increased maturity is desired, the invention teaches that inhibition of NR2F6 activity is to be performed. Conversely, when inhibition of differentiation of said stem cell is desired, said inhibition of differentiation is achieved through induction of increased NR2F6 activity. Within the context of the invention, the word “stem cell” may also mean a progenitor cell. In one embodiment of the invention, stem cell is selected from a group comprising of embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Particularly, for the practice of the invention, said embryonic stem cells are totipotent and express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Cord blood stem cells are multipotent and capable of differentiating into endothelial, smooth muscle, hematopoietic and neuronal cells. Wherein, said cord blood stem cells are identified based on expression of one or more antigens selected from a group comprising: SSEA-3, SSEA-4, CD9, CD34, c-kit, OCT-4, Nanog, and CXCR-4. Furthermore, for the practice of the invention, said cord blood stem cells express the marker CD34. When placental stem cells are utilized, said cells are isolated from the placental structure and are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2. Furthermore, within the context of the current invention bone marrow stem cells may be used. Said cells may be utilized as purified hematopoietic stem cells, or may be bone marrow derived mononuclear cells. In a preferred embodiment, CD34 cells isolated from the bone marrow are utilized. Said bone marrow stem cells possess the possibility to differentiate into one or more of the following cell types: endothelial cells, smooth muscle cells, and neuronal cells, as well as along the hematopoietic lineage. In a preferred embodiment hematopoietic lineage cells are utilized. Said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

The technique of bone marrow harvesting is well-known in the art. In one embodiment, bone marrow harvest is performed with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest” (Mineishi, S. 2003. Nippon Rinsho 61:1489-1494, which is incorporated by reference herein in its entirety). Said aspirate is used as a starting material for purification of cells with angiogenic activity. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS300-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In general, apheresis is used to isolate cellular components from the blood and involves removing blood from a subject, subjecting the blood to a separation method to remove certain components, and reinfusing the blood into the subject in a continuous manner.

In another embodiment placental stem cells are isolated from the Wharton's Jelly, with said placental stem cells being mesenchymal in morphology and expressing one or more cytokines associated with a regenerative activities. Cytokines associated with regenerative activity as selected from a group comprising of CS-6, IL-6, IL-8, SDF-1, CXCL5, VEGF, CXCL6, COL4A4, MMP13, CYP7B1, ADAMDEC1, SLC6A1, CXCL1, PF4V1, CXCL3, CH25H, SFRP2, MMP1, DARC, HCK, bFGF, ERC2, CLIC6, and BCL8 in a 75% confluent culture. Said placental stem cells express between 200.0-1900.0 pg/ml of IL-6 in a 75% confluent culture; between 350.0-2,000.0 pg/ml of bFGF in a 75% confluent culture; between 500.0-2,500.0 pg/ml of VEGF in a 75% confluent culture; and between 140.0-1,500.0 pg/ml of SDF-1 in a 75% confluent culture.

In one embodiment of the invention adipose stem cells are utilized, wherein said adipose stem cell expresses markers selected from a group comprising of: CD13, CD29, CD44, CD63, CD73, CD90, CD166, Aldehyde dehydrogenase (ALDH), and ABCG2. Said adipose tissue derived stem cells are a population of purified mononuclear cells extracted from adipose tissue capable of proliferating in culture for more than 1 month.

In one embodiment of the invention, side population stem cells are utilized, said side population stem cells may be purified based on tissue dissociation and selection for cells expressing rhodamin 123 efflux properties. Said purification may be performed by enzymatic dissociation and flow sorting. Said side population cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In one embodiment of the invention stem cells are utilized that have been immortalized. Immortalization methods are known in the art and may include transfection, either permanent or inducible with oncogenes. Known oncogenes that have been utilized for the purposes of immortalization include telomerase, or may be selected from a group comprising of: a) abl; b) Af4/hrx; c) akt-2; d) alk; e) alk/npm; 0 amll; g) amll/mtg8; h) bcl-2, 3, 6; i) bcr/abl; j) c-myc; k) dbl; l) dek/can; m) E2A/pbx1; n) egfr; o) enl/hrx; p) erg/TLS; q) erbB; r) erbB-2; s) ets-1; t) ews/fli-1; u) fms; v) fos; w) fps; x) gli; y) gsp; z) gsp; aa) HER2/new; ab)hox11; ac) hst; ad) IL-3; ae) int-2; af) jun; ag) kit; ah) KS3; ai) K-sam; aj)Lbc; ak) lck; al) Imol, Imo-2; am) L-myc; an) lyl-1; ao) lyt-10; ap)lyt-10/C alpha 1; aq) mas; ar) mdm-2; as) mll; at) mos; au) mtg8/am11; av) myb; aw) MYH11; ax) new; ay) N-myc; az) ost; ba) pax-5; bb) pbx1/E2a; bc) pim-1; bd) PRAD-1; be) raf; bf) RAR/PML; bg) Ras H, K, N; bh) rel/nrg; bi) ret; bj) rhoml, rhom2; bk) ros; bl) ski; bm) sis; bn) set/can; bo) src; bp) Tal1, tal2; bq) tan-1; br) Tiam1; bs) TSC2; and bt) trk.

NR2F6 activity may be suppressed by treatment of stem cells with an oligonucleotide capable of inhibiting expression of NR2F6 gene. Said inhibition may be obtained as a result of induction of an antisense mediated inhibition of mRNA or may be achieved by induction of RNA interference. Specifically, a oligonucleotide comprising of a short-interfering ribonucleic acid (siRNA) molecule. SEQ IDs NO: 9-12 or 16-17 comprise nucleic acid sequences that may be used as one part of the double stranded RNA or single stranded oligonucleotides for induction of RNAi or antisense mediated inhibition. In one particular embodiment the invention teaches the enhancement of hematopoietic stem cell differentiation of a mammalian stem cell comprising, transfecting said stem cells with an oligonucleotide capable of suppressing expression of NR2F6. Said suppression of expression may be achieved by transfected with an oligonucleotide in which one strand possesses at least 75% homology with SEQ ID NO: 9 -12 or 16-17. In one particular embodiment stem cells are treated with an oligonucleotide complementary to a nuclear receptor having a mRNA sequence of at least 75% sequence identity to the mRNA sequence of SEQ ID NO: 5, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that is selected based on its ability to inhibits the expression of the nuclear receptor NR2F6 by causing degradation of a ribonucleic acid encoding nuclear receptor NR2F6. Said stem cells are hematopoietic stem cells in one embodiment of the invention, said stem cells being autologous or allogeneic. Patients may be treated in which accelerated differentiation is desired such as a patient suffering from, or who is susceptible to, decreased blood cell levels. Said decreased blood cell levels may be caused by chemotherapy, radiation therapy, bone marrow transplantation therapy or congenital anemia. In another embodiment the invention teaches a method of treating a mammal in need of improved hematopoietic capability, comprising the steps of: (a) removing hematopoietic stem cells from the mammal; (b) transfecting said stem cells with an oligonucleotide capable of suppressing expression of NR2F6; (c) culturing said transfected stem cells to form an expanded population of stem cells; and (d) returning said expanded cells to the mammal, whereby hematopoietic capability is improved.

The term “NR2F6” as used herein refers to nuclear receptor subfamily2, group F, member 6 and is also referred to as v-erbA-related gene or ear-2 and includes, without limitation, the protein encoded by the gene having the sequence as shown in SEQ ID NO:5 (human) or SEQ ID NO: 6 (mouse) or variants thereof and the protein having the amino acid sequence as shown in SEQ ID NO: 7 (human) or SEQ ID NO:8 (mouse) or variants thereof. Variants thereof include alternatively spliced variants, or variants of the domains found in this gene that have 75% sequence homology or greater at the protein level, that are present in other genes.

The term “a cell” as used herein includes a plurality of cells and refers to all types of cells including hematopoietic stem cells, tissue specific stem cells and cancer cells. Administering a compound to a cell includes in vivo, ex vivo and in vitro treatment.

The term “stem cell” as used herein refers to a cell that has the ability for self-renewal. Non-cancerous stem cells have the ability to differentiate where they can give rise to specialized cells.

The term “effective amount” as used herein means a quantity sufficient to, when administered to an animal, effect beneficial or desired results, including clinical results, and as such, an “effective amount” depends upon the context in which it is being applied. For example, in the context of inhibiting self-renewal of stem cells, it is the amount of the NR2F6 inhibitor sufficient to achieve such an inhibition as compared to the response obtained without administration of the NR2F6 inhibitor.

The term “oligonucleotide” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “animal” as used herein includes all members of the animal kingdom, preferably mammal. The term “mammal” as used herein is meant to encompass, without limitation, humans, domestic animals such as dogs, cats, horses, cattle, swine, sheep, goats, and the like, as well as wild animals. In an embodiment, the mammal is human.

The term “interfering RNA” or “RNAi” or “interfering RNA sequence” refers to double-stranded RNA (i.e., duplex RNA) that targets (ie., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in:length). siRNA duplexes may comprise 3′ overhangs of about 1 to about 4 nucleotides, preferably of about 2 to about 3 nucleotides and 5′ phosphate termini, The siRNA can be chemically synthesized or maybe encoded by a plasmid (e.g., transcribed as sequences that automatically fold into duplexes with hairpin loops), siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99; 9942-7 (2002); Calegari et al., PNAS USA 99: 14236 (2002); Byrom et al. Ambion TechNotes 10(1): 4-6 (2003); Kawasaki et al., Nucleic Acids Res. 31: 981-7 (2003); Knight and Bass, Science 293: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length, or longer. The dsRNA can encode for an entire gene transcript or a partial gene transcript,

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

The term “antisense nucleic acid” as used herein means a nucleotide sequence that is complementary to its target e.g. a NR2F6 transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

As used in this context, to “treat” means to ameliorate at least one symptom of the disorder. In some embodiments, a treatment can result in a reduction in tumor size or number, or a reduction in tumor growth or growth rate.

One embodiment of the invention is a short-interfering ribonucleic acid (siRNA) molecule effective at silencing NR2F6 expression or substantially inhibiting NR2F6 expression. In one embodiment of the invention the oligonucleotide backbone is chemically modified to increase the deliverability of the interfering ribonucleic acid molecule. In another embodiment these chemical modifications act to neutralize the negative charge of the interfering ribonucleic acid molecule. One embodiment of the invention consists of a pharmaceutical composition comprising an siRNA oligonucleotide that induces RNA interference against NR2F6. It is known to one of skill in the art that siRNAs induce a sequence-specific reduction in expression of a gene by the process of RNAi, as previously mentioned. Thus, siRNA is the intermediate effector molecule of the RNAi process that is normally induced by double stranded viral infections, with the longer double stranded RNA being cleaved by naturally occurring enzymes such as DICER. Some nucleic acid molecules or constructs provided herein include double stranded RNA molecules comprising 16-30, e.g., 16, 17, 18, 19, 20, 21, 22, 23, 24, 25, 26, 27, 28, 29, or 30 nucleotides in each strand, wherein one of the strands is substantially identical, for example at least 85% (or more, as for example, 90%, 95%, or 100%) identical, e.g., having 3, 2, 1, or 0 mismatched nucleotide(s), to a target region in the mRNA of NR2F6 and the other strand is identical or substantially identical to the first strand. However, it will be appreciated that the dsRNA molecules may have any number of nucleotides in each strand which allows them to reduce the level of NR2F6 protein, or the level of a nucleic acid encoding NR2F6. The dsRNA molecules provided herein can be chemically synthesized, or can be transcribed in vitro from a DNA template, or in vivo from, e.g., shRNA, which is mentioned below. The dsRNA molecules can be designed using any method known in the art.

In one embodiment, nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single cros slink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F6. In some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F6 gene, such as a nucleic acid encoding the NR2F6 mRNA, and can be driven, for example, by separate Pol III promoter sites. In some situations it will be preferable to induce expression of the hairpin siRNA or shRNAs in a tissue specific manner in order to activate the shRNA transcription that would subsequently silence NR2F6 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues. Examples of more tissue specific promoters include in (a) to target the pancreas promoters for the following may be used: insulin, elastin, amylase, pdr-I, pdx-I, glucokinase; (b) to target the liver promoters for the following may be used: albumin PEPCK, HBV enhancer, a fetoprotein, apolipoprotein C, .alpha.-I antitrypsin, vitellogenin, NF-AB, Transthyretin; (c) to target the skeletal muscle promoters for the following may be used: myosin H chain, muscle creatine kinase, dystrophin, calpain p94, skeletal .alpha.-actin, fast troponin 1; (d) to target the skin promoters for the following may be used: keratin K6, keratin KI; (e) lung: CFTR, human cytokeratin IS (K 18), pulmonary surfactant proteins A, B and C, CC-10, Pi; (0 smooth muscle: sm22 .alpha., SM-.alpha.-actin; (g) to target the endothelium promoters for the following may be used: endothelin-I, E-selectin, von Willebrand factor, TIE, KDR/flk-I; (h) to target melanocytes the tyrosinase promoter may be used; (i) to target the mammary gland promoters for the following may be used: MMTV, and whey acidic protein (WAP).

Yet another embodiment of the invention consists of a pharmaceutical composition comprising an oligonucleotide that induces RNA interference against NR2F6 combined with a delivery agent such as a liposome. For more targeted delivery immunoliposomes, or liposomes containing an agent inducing selective binding to neoplastic cells may be used.

The present invention further provides pharmaceutical compositions comprising the nucleic acid-lipid particles described herein and a pharmaceutically acceptable carrier.

Another embodiment of the invention consists of a pharmaceutical composition comprising an oligonucleotide that induces RNA interference against NR2F6 combined with an additional chemotherapeutic agent.

Yet another embodiment of the invention consists of a pharmaceutical composition comprising an oligonucleotide that induces RNA interference against NR2F6 combined with an additional agent used to induce differentiation

One embodiment of the invention is a short-interfering ribonucleic acid (siRNA) molecule effective at silencing NR2F6 expression that has been cloned in to an appropriate expression vector giving rise to an shRNA vector.

In certain embodiment shRNA olignucleotides are cloned in to an appropriate mammalian expression vectors, examples of appropriate vectors include but are not limited to lentiviral, retroviral or adenoviral vector.

In this embodiment, the invention consists of a viral vector, comprising the inhibitory RNA molecule described above. The viral vector preferably is a lentivirus. In one aspect the viral vector is capable of infecting stem cells. Another embodiment is a lentivirus vector that is an integrating vector. The viral vector preferably is capable of transducing stem cells. The viral vector is preferably packaged in a coat protein the specifically binds to stem cells. The viral vector preferably is capable of expressing an RNA that inhibits NR2F6 expression. Another embodiment of the invention is one in which the viral vector is preferably produced by a vector transfer cassette and a separate helper plasmid. In certain embodiment the shRNA olignucleotides is combined with a pharmaceutically acceptable vehicle a pharmaceutical composition. One embodiment is a pharmaceutical composition comprising an inhibitory oligonucleotide that is a double stranded RNA molecule.

One aspect of the invention is a microRNA or family of microRNAs are administered that substantially inhibit expression of NR2F6

Accordingly, the present disclosure provides a method of inhibiting self-renewal of stem cells comprising administering an effective amount of an oligonucleotides that induce RNA interference to a cell or animal in need thereof. The present disclosure also provides the use of a oligonucleotides that induce RNA interference for inhibiting self-renewal of stem cells in a cell or animal in need thereof. The present disclosure further provides the use of an oligonucleotide that induce RNA interference in the preparation of a medicament for inhibiting self-renewal of stem cells in a cell or animal in need thereof. The present disclosure also provides a oligonucleotides that induce RNA interference for use in inhibiting self-renewal of stem cells in a cell or animal in need thereof.

In another embodiment, the present disclosure provides a method of inducing terminal differentiation of stem cells comprising administering of an effective amount of oligonucleotides that induce RNA interference to NR2F6 to a cell or animal in need thereof. The present disclosure also provides the use of oligonucleotides that induce RNA interference to NR2F6 for inducing terminal differentiation of stem cells in a cell or animal in need thereof. The present disclosure further provides the use of oligonucleotides that induce RNA interference to NR2F6 in the preparation of a medicament for inducing terminal differentiation of stem cells in a cell or animal in need thereof. The present disclosure also provides oligonucleotides that induce RNA interference to NR2F6 for use in inducing terminal differentiation of stem cells in a cell or animal in need thereof.

In one embodiment, the stem cells are hematopoietic stem cells, leukemia stem cells or ovarian cancer stem cells.

The term “inhibiting self renewal of stem cells” as used herein includes but is not limited to preventing or decreasing the clonal longevity, clonogenicity, serial replating ability, clonogenic growth and/or transplantability of the stem cells.

In one embodiment the invention provides a method for enhancing the hematopoietic differentiation of mammalian stem cells, with particular propensity towards the T cell lineage, and also erythroid lineage. The method is useful for generating expanded populations of hematopoietic stem cells (HSCs) and thus mature blood cell lineages. This is desirable where a mammal has suffered a decrease in hematopoietic or mature blood cells as a consequence of disease, radiation, chemotherapy or congenital anemia (ie, Diamond Blackfan Anemia).

In one embodiment present invention comprises decreasing the intracellular level of a NR2F6 in stem cells, including hematopoietic stem cells, in culture, either by providing an exogenous mediator capable of reducing mRNA encoding for NR2F6 protein to the cell, or by introduction into the cell of a double stranded RNA capable of inducing RNAi to suppress NR2F6 transcripts.

In one embodiment differentiated and expanded cell populations are useful as a source of hematopoietic stem cells, which may be used in transplantation to restore hematopoietic function to autologous or allogeneic recipients.

The invention provides means of inducing differentiation of stem cells through inhibition of the expression and/or activity of the NR2F6 gene and/or protein respectively. In one aspect, induction of differentiation is performed by administration of an agent or plurality of agents capable of inhibiting expression of the NR2F6 gene. Said means of inhibition include administration of hammerhead ribozymes, gene editing means such as TALON or CRISPER mediated DNA cleavage, or means capable of inducing RNA interference such as short interfering RNA (siRNA) or induction of DNA directed RNA interference such as short hairpin RNA (shRNA) expressed from a plasmid, viral, or lentiviral vector. Additionally, inhibition of gene activity may be obtained by administration of antisense oligonucleotides. The invention discloses compositions comprising synthetic oligonucleotide molecules that induce RNA interference of the NR2F6 gene, and methods of treating cancer by blocking expression of the gene NR2F6 using synthetic oligonucleotides that induce RNA interference. The RNA interference inducing oligonucleotide is one of the following: short interfering nucleic acid (siNA), short interfering RNA (siRNA), double-stranded RNA (dsRNA), micro-RNA (miRNA), and short hairpin RNA (shRNA) molecules.

In one embodiment of the invention, the RNAi inducing oligonucleotides are targeted to the stem cells by introduction of an exogenous nucleic acid expression vector into the cells. Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, or ALV. Various techniques known in the art may be used to transfect the target cells, e.g. electroporation, calcium precipitated DNA, fusion, transfection, lipofection and the like. The particular manner in which the DNA is introduced is not critical to the practice of the invention. Combinations of retroviruses and an appropriate packaging line may be used, where the capsid proteins will be functional for infecting the target cells. Usually, the cells and virus will be incubated for at least about 24 hours in the culture medium. In some situations it is desirable to induce expression of gene silencing oligonucleotides, this may be performed by inserting a short hairpin siRNA under control of an inducible promoter. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in hematopoietic cell types, e.g. IL-2 promoter in T cells or immunoglobulin promoter in B cells. In an alternative method, expression vectors that provide for the transient expression in mammalian cells may be used. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient short term expansion of cells, but do not affect the long term genotype of the cell.

In one embodiment of the invention, tat protein is used to deliver siRNA or shRNA. The optimal transport polypeptides are characterized by the presence of the tat basic region amino acid sequence (amino acids 49-57 of naturally-occurring tat protein); the absence of the tat cysteine-rich region amino acid sequence (amino acids 22-36 of naturally-occurring tat protein) and the absence of the tat exon 2-encoded carboxy-terminal domain (amino acids 73-86 of naturally-occurring tat protein).

In one embodiment NR2F2 or NR2F1 is utilized as a target instead of NR2F6 to achieve the purpose of the invention, that is, to stimulate differentiation of stem cells.

Similarly, in one embodiment NR2F2 or NR2F1 or NR2F6 or a combination, is silenced or substantially inhibited as a means of stimulating T cell differentiation. In another embodiment NR2F2 or NR2F1 or NR2F6 or a combination thereof are overexpressed as a means of blocking T cell differentiation of hematopoietic stem cells.

### Sequence Listings

Sequence IDs for inhibition of NR2F2 are

Human NR2F2 siRNA 1
SEQ ID No: 1
GCCGUCUCAAGAAGUGCUU
Human NR2F2 siRNA 2
SEQ ID No: 2
CAUUGAGACACUGAUCAGA
Human NR2F2 siRNA 3
SEQ ID No: 3
GCAAGCAUUACGGUGUCUU
Human NR2F2 siRNA 4
SEQ ID No: 4
CCCCUAGCAUGAACUUGUG
NCBI Reference Sequence: NM_005234.3
>gi|46411186|ref|NM_005234.31 Homo
sapiens nuclear receptor subfamily 2,
group F, member 6 (NR2F6), mRNA
SEQ ID No: 5
GTGCAGCCCGTGCCCCCCGCGCGCCGGGGCCGAATGCGCGCCGCGTA
GGGTCCCCCGGGCCGAGAGGGGTGCCCGGAGGGAAGAGCGCGGTGGG
GGCGCCCCGGCCCCGCTGCCCTGGGGCTATGGCCATGGTGACCGGCG
GCTGGGGCGGCCCCGGCGGCGACACGAACGGCGTGGACAAGGCGGGC
GGCTACCCGCGCGCGGCCGAGGACGACTCGGCCTCGCCCCCCGGTGC
CGCCAGCGACGCCGAGCCGGGCGACGAGGAGCGGCCGGGGCTGCAGG
TGGACTGCGTGGTGTGCGGGGACAAGTCGAGCGGCAAGCATTACGGT
GTCTTCACCTGCGAGGGCTGCAAGAGCTTTTTCAAGCGAAGCATCCG
CCGCAACCTCAGCTACACCTGCCGGTCCAACCGTGACTGCCAGATCG
ACCAGCACCACCGGAACCAGTGCCAGTACTGCCGTCTCAAGAAGTGC
TTCCGGGTGGGCATGAGGAAGGAGGCGGTGCAGCGCGGCCGCATCCC
GCACTCGCTGCCTGGTGCCGTGGCCGCCTCCTCGGGCAGCCCCCCGG
GCTCGGCGCTGGCGGCAGTGGCGAGCGGCGGAGACCTCTTCCCGGGG
CAGCCGGTGTCCGAACTGATCGCGCAGCTGCTGCGCGCTGAGCCCTA
CCCTGCGGCGGCCGGACGCTTCGGCGCAGGGGGCGGCGCGGCGGGCG
CGGTGCTGGGCATCGACAACGTGTGCGAGCTGGCGGCGCGGCTGCTC
TTCAGCACCGTGGAGTGGGCGCGCCACGCGCCCTTCTTCCCCGAGCT
GCCGGTGGCCGACCAGGTGGCGCTGCTGCGCCTGAGCTGGAGCGAGC
TCTTCGTGCTGAACGCGGCGCAGGCGGCGCTGCCCCTGCACACGGCG
CCGCTACTGGCCGCCGCCGGCCTCCACGCCGCGCCTATGGCCGCCGA
GCGCGCCGTGGCTTTCATGGACCAGGTGCGCGCCTTCCAGGAGCAGG
TGGACAAGCTGGGCCGCCTGCAGGTCGACTCGGCCGAGTATGGCTGC
CTCAAGGCCATCGCGCTCTTCACGCCCGACGCCTGTGGCCTCTCAGA
CCCGGCCCACGTTGAGAGCCTGCAGGAGAAGGCGCAGGTGGCCCTCA
CCGAGTATGTGCGGGCGCAGTACCCGTCCCAGCCCCAGCGCTTCGGG
CGCCTGCTGCTGCGGCTCCCCGCCCTGCGCGCGGTCCCTGCCTCCCT
CATCTCCCAGCTGTTCTTCATGCGCCTGGTGGGGAAGACGCCCATTG
AGACACTGATCAGAGACATGCTGCTGTCGGGGAGTACCTTCAACTGG
CCCTACGGCTCGGGCCAGTGACCATGACGGGGCCACGTGTGCTGTGG
CCAGGCCTGCAGACAGACCTCAAGGGACAGGGAATGCTGAGGCCTCG
AGGGGCCTCCCGGGGCCCAGGACTCTGGCTTCTCTCCTCAGACTTCT
ATTTTTTAAAGACTGTGAAATGTTTGTCTTTTCTGTTTTTTAAATGA
TCATGAAACCAAAAAGAGACTGATCATCCAGGCCTCAGCCTCATCCT
CCCCAGGACCCCTGTCCAGGATGGAGGGTCCAATCCTAGGACAGCCT
TGTTCCTCAGCACCCCTAGCATGAACTTGTGGGATGGTGGGGTTGGC
TTCCCTGGCATGATGGACAAAGGCCTGGCGTCGGCCAGAGGGGCTGC
TCCAGTGGGCAGGGGTAGCTAGCGTGTGCCAGGCAGATCCTCTGGAC
ACGTAACCTATGTCAGACACTACATGATGACTCAAGGCCAATAATAA
AGACATTTCCTACCTGCA
Mus musculus nuclear receptor subfamily 2,
group F, member 6 (Nr2f6), mRNA NCBI Reference
Sequence: NM_010150.2
>gi|112807198|ref|NM_010150.2|Mus musculus
nuclear receptor subfamily 2, group F, member 6
(Nr2f6), mRNA
Sequence ID No: 6
GCGCCGATGGAACGCGGGTGTCAGGCCGGCCGCAGCGCGGGGCCGGC
GGCGAGCGCCAGGGCGAGGCCGAGGCTCGGGCCCAGGCGCAGGCCGA
GGCCGGCCGCGCGAGCGCTCGGCGGGGAGACGATCCAGGGAAGGCCG
CGGGTCGCACTCTCCACTCAGCTCTATCGCCTGGACCTCTGCGATTA
CGGCCGGGCGCGCGCGGCGTGCGGGACTCCGGGTCTCCGACGCGCGC
TCCCGCCGCCCCTCCCCCCTCGCCGCGTAACTTGCGGCCAAAGTTTC
CCCCCGGGCTCGGGGGCGCCCGCGCGCGCTCGGATGGTGAGCCACTA
AGTTGGCCTGGGCGGCGGGGCCGGGCCATGGCCCCCGCGACGCTACC
GGGTCCCCAGGACTCCGGACCACGGGACCTGGGCGCCCCAGACTCGC
GCCTCTAGCGCGCCCCCGTCGACCGCGGGCACGCGTGGGAAAGTTGG
CCTGGAACCGGCCCGACCAGTTCCTGCCTGGCGCGCGGACCGGCCGC
AGGAAGTTGCCGCAAAACTTTTTTCAGGGGGGTGTGCGACCGGAGCC
CCCCGAGAGCGCGGGCTGCATGCGCCCGGGGTAGCCGGGTCCCTCTC
GGGTCGCCAGGCGTGCCCAGAGGGGACGGACTCGTCCCGGGGCGTAC
CGGCCCCGCTGTCTCCGGGGCTATGGCCATGGTGACCGGTGGCTGGG
GCGACCCCGGAGGCGACACGAACGGCGTGGACAAGGCTGGTGGGAGC
TACCCACGCGCGACCGAGGACGATTCGGCGTCACCTCCCGGGGCGAC
CAGCGACGCGGAGCCGGGCGACGAGGAGCGTCCGGGGTTGCAGGTGG
ACTGCGTGGTGTGCGGGGACAAGTCCAGTGGAAAGCATTACGGCGTG
TTCACCTGCGAGGGCTGCAAGAGTTTCTTCAAGCGCAGCATCCGCCG
CAATCTCAGCTACACCTGCCGGTCCAACCGTGACTGTCAGATTGATC
AGCACCACCGGAACCAGTGTCAGTACTGTCGGCTCAAGAAGTGCTTC
CGGGTGGGCATGCGCAAGGAGGCCGTGCAGCGAGGCCGCATCCCGCA
TGCGCTCCCCGGTCCAGCGGCCTGCAGTCCCCCGGGCGCGACGGGCG
TCGAACCTTTCACGGGGCCGCCAGTGTCCGAGCTGATTGCGCAGCTG
CTGCGTGCTGAGCCCTACCCCGCGGCCGGACGCTTTGGTGGCGGCGG
CGCTGTACTGGGCATCGACAACGTGTGCGAGTTGGCGGCACGCCTGC
TGTTCAGCACGGTCGAGTGGGCCCGCCACGCGCCCTTCTTCCCCGAG
CTGCCGGCCGCCGACCAGGTGGCGCTGCTGCGGCTCAGCTGGAGTGA
GCTCTTCGTGCTGAACGCGGCGCAGGCGGCGCTGCCGCTGCATACGG
CACCGCTGCTGGCCGCCGCGGGGTTGCATGCCGCGCCCATGGCAGCC
GAGCGGGCCGTGGCCTTCATGGACCAGGTGCGTGCCTTCCAGGAGCA
GGTGGACAAGCTGGGCCGCCTGCAGGTGGATGCTGCGGAGTACGGCT
GCCTCAAGGCCATCGCGCTCTTCACGCCTGATGCCTGTGGCCTTTCT
GACCCAGCCCATGTGGAGAGCCTGCAGGAGAAGGCACAGGTGGCCCT
CACCGAGTATGTGCGTGCCCAGTACCCATCGCAGCCCCAGCGCTTTG
GGCGTCTGCTGCTGCGGCTGCCAGCCCTGCGTGCTGTGCCCGCATCC
CTCATCTCCCAGCTCTTCTTCATGCGCCTGGTGGGCAAGACACCCAT
CGAGACCCTCATCCGGGACATGCTTCTGTCAGGGAGCACCTTTAACT
GGCCCTATGGCTCGGGCTAGTGATAGTCACCTTCCAGGACATACATG
GAAACTGGGGCCTTGTGGGGACCCTGGGGATCAGGGCCCCAGCTTCT
CTTTTGAGACTGATTTCTTTTTTTAAAGACTGTGAAATGTTTGTTTT
GTTTTATTTTTTAAATAATCATGAAACCAAAAAGATTTGGATCTCCC
AGGCCTTGTCCTGGCAGACCTTCAACAGTCTGGAGCCAGCATGCTGA
TGCCTCTGGTGTCATGGGTATCTGGAAAGGCCACTGCAGCTAGGCAG
GAGTACTATGGGCCAGGAGGATCCCCTGGATACATGGTCCACGGAGG
GCACCATGGGATGATGAAAACCTGGCCAATAATAAAGGTATTCCCTT
ACTTGGTC
Protein Sequence of human NR2F6
>gi|23503053|sp|P10588.2|NR2F6_HUMAN
RecName: Full = Nuclear receptor subfamily
2 group F member 6; AltName: Full = V-erbA-
related protein 2; Short = EAR-2
SEQ ID No: 7
MAMVTGGWGGPGGDTNGVDKAGGYPRAAEDDSASPPGAASDAEPGDE
ERPGLQVDCVVCGDKSSGKHYGVFTCEGCKSFFKRSIRRNLSYTCRS
NRDCQIDQHHRNQCQYCRLKKCFRVGMRKEAVQRGRIPHSLPGAVAA
SSGSPPGSALAAVASGGDLFPGQPVSELIAQLLRAEPYPAAAGRFGA
GGGAAGAVLGIDNVCELAARLLFSTVEWARHAPFFPELPVADQVALL
RLSWSELFVLNAAQAALPLHTAPLLAAAGLHAAPMAAERAVAFMDQV
RAFQEQVDKLGRLQVDSAEYGCLKAIALFTPDACGLSDPAHVESLQE
KAQVALTEYVRAQYPSQPQRFGRLLLRLPALRAVPASLISQLFFMRL
VGKTPIETLIRDMLLSGSTFNWPYGSGQ
Protein Sequence of NR2F6 mus musculus
>gi|112807199|ref|NP_034280.2|nuclear receptor
subfamily 2 group F member 6 [Mus musculus]
SEQ ID No: 8
MAMVTGGWGDPGGDTNGVDKAGGSYPRATEDDSASPPGATSDAEPGD
EERPGLQVDCVVCGDKSSGKHYGVFTCEGCKSFFKRSIRRNLSYTCR
SNRDCQIDQHHRNQCQYCRLKKCFRVGMRKEAVQRGRIPHALPGPAA
CSPPGATGVEPFTGPPVSELIAQLLRAEPYPAAGRFGGGGAVLGIDN
VCELAARLLFSTVEWARHAPFFPELPAADQVALLRLSWSELFVLNAA
QAALPLHTAPLLAAAGLHAAPMAAERAVAFMDQVRAFQEQVDKLGRL
QVDAAEYGCLKAIALFTPDACGLSDPAHVESLQEKAQVALTEYVRAQ
YPSQPQRFGRLLLRLPALRAVPASLISQLFFMRLVGKTPIETLIRDM
LLSGSTFNWPYGSG
(human siRNA)
SEQ ID NO: 9
GCCGUCUCAAGAAGUGCUU
(human siRNA)
SEQ ID NO: 10
CAUUGAGACACUGAUCAGA
(human siRNA)
SEQ ID NO: 11
GCAAGCAUUACGGUGUCUU
(human siRNA)
SEQ ID NO: 12
CCCCUAGCAUGAACUUGUG
(mus shNR2F6.1)
SEQ ID NO: 13
GAT CCG CAT TAC GGC GTG TTC ACC TTC AAG AGA GGT
GAA CAC GCC GTA ATG CTT TTT TCT AGA G
(mus shNR2F6.2)
SEQ ID NO: 14
GAT CCG CAA CCG TGA CTG TCA GAT TAA GTT CTC TAA
TCT GAC AGT CAC GGT TGT TTT TTC TAG AG
(mus shNR2F6.3)
SEQ ID NO: 15
GAT CCG TGT CCG AGC TGA TTG CGC ATT CAA GAG ATG
CGC AAT CAG CTC GGA CAT TTT TTC TAG AG
(human shNR2F6.1)
SEQ ID NO: 16
GAT CCG CAT TAC GGT GTC TTC ACC TTC AAG AGA GGT
GAA GAC ACC GTA ATG CTT TTT TCT AGA G
(human shNR2F6.2)
SEQ ID NO: 17
GAT CCG CCT CTG GAC ACG TAA CCT ATT CAA GAG ATA
GGT TAC GTG TCC AGA GGT TTT TTC TAG AG
Primers
Human NR2F6 Forward Primer:
SEQ ID NO: 18
Fwd: 5′-TCTCCCAGCTGTTCTTCATGC-3′
Human NR2F6 Reverse Primer:
SEQ ID NO: 19
Revs: 5′-CCAGTTGAAGGTACTCCCCG-3′
Human GAPDH Forward Primer:
SEQ ID NO: 20
Fwd: 5′-GGCCTCCAAGGAGTAAGACC-3′
Human GAPDH Reverse Primer:
SEQ ID NO: 21
Revs: 5′-AGGGGTCTACATGGCAACTG-3′.
3′ end Mus NR2F6 Forward Primer:
SEQ ID NO: 22
Fwd: 5′-CCTGGCAGACCTTCA ACAG-3′
3′ end Mus NR2F6 Reverse Primer:
SEQ ID NO: 23
Revs: 5′-GATCCTCCTGGCCCATAGT-3′
3′ end Mus L32 Forward Primer:
SEQ ID NO: 24
Fwd: 5′-GCCATCAGAGTCACCAATCC-3′
3′ end Mus L32 Reverse Primer:
SEQ ID NO: 25
Revs: 5′-AAACATGCACACAAGCCATC-3′

List of compounds useful for blocking NR2F2 include the following and variations thereof:

| Compound name | Molecular weight |
|---|---|
| MLS003122208 | 487.634 |
| AC1NUNJE | 419.429 |
| Ossamycin | 912.197 |
| SMR000124769 | 498.617 |
| MLS000735463 | 333.471 |
| MLS002702449 | 557.583 |
| SMR000064686 | 374.887 |
| Dihydrorotenone | 396.433 |
| 3-(Toluene-4-sulfonylmethyl)-2,3- | 344.451 |
| dihydro-benzo[4,5]imidazo[2,1-b]thiazole | |
| SMR000185185 | 356.256 |
| SMR000093473 | 415.479 |
| Ambcb87269753 | 395.408 |
| ZINC03428816 | 362.469 |
| AC1NM0NL | 500.009 |
| Ambcb20390854 | 395.451 |
| ISUPSL100073 | 384.423 |
| ZINC00523887 | 329.396 |
| ACTIPHENOL | 275.299 |
| MLS003120814 | 484.592 |
| Ambcb55536813 | 363.456 |
| T6151837 | 490.956 |
| T6094971 | 413.877 |
| MLS003119103 | 412.458 |
| SMR000121626 | 490.964 |
| F0239-0029 | 290.231 |
| SMR000673572 | 299.754 |
| SMR000629820 | 475.539 |
| T6099016 | 494.627 |
| MLS003120011 | 420.414 |
| AC1LCZCI | 300.357 |
| 4-[4-(1,3-benzodioxol-5- | 417.46 |
| ylmethyl)piperazin-1-yl]-7-methoxy-5H- | |
| pyrimido[5,4-b]indole | |
| AGN-PC-00PL3I | 369.819 |
| AGN-PC-03RL0E | 374.41 |
| T5400648 | 378.447 |
| SMR000635220 | 529.093 |
| T5573980 | 400.54 |
| T5546966 | 372.644 |
| MLS003128611 | 482.56 |
| MLS003120807 | 470.566 |
| 2-(5-Pyridin-3-yl-[1,3,4]thiadiazol-2- | 379.458 |
| ylsulfanyl)-N-quinolin-4-yl-acetamide | |
| MLS000575323 | 425.954 |
| MLS001028777 | 433.545 |
| T5235682 | 417.866 |
| MLS002477203 | 484.592 |
| MLS001105915 | 419.476 |
| Pyridaben | 364.932 |
| MLS002701851 | 297.262 |
| Ambcb33735952 | 401.457 |
| SMR000003660 | 474.51 |
| MLS002473640 | 353.438 |
| MLS000733369 | 379.452 |
| AC1OBZ0O | 404.458 |
| SMR000218920 | 465.329 |
| N-methyl-N-[(1,3,5-trimethyl-1H-pyrazol- | 343.443 |
| 4-yl)methyl]-2-naphthalenesulfonamide | |
| AC1MGVNO | 380.466 |
| T5459762 | 327.417 |
| SMR000017708 | 475.537 |
| N-[2-(3,4-dimethoxyphenyl)ethyl]-6- | 365.877 |
| methylthieno[2,3-d]pyrimidin-4-amine | |
| hydrochloride | |
| N-(1-phenylethyl)quinazolin-4-amine | 249.31 |
| cycloheximide | 281.347 |
| Streptovitacin | 297.346 |
| T0503-0850 | 437.395 |
| MLS000688479 | 474.4 |
| AGN-PC-00YPMB | 431.435 |
| MLS000562030 | 591.999 |
| SMR000285129 | 502.454 |
| MLS000037490 | 307.391 |
| AC1N4L8I | 406.538 |
| MLS000586514 | 296.363 |
| SMR000212173 | 403.335 |
| T0503-4033 | 433.34 |
| ASN 09858385 | 477.555 |
| MLS003120821 | 524.588 |
| MLS002548992 | 329.439 |
| MLS001198897 | 300.357 |
| SMR000629835 | 489.566 |
| AC1MLRO7 | 427.539 |
| Brusatol | 520.525 |
| AC1LEXWX | 301.181 |
| 4-ethoxy-N-(pyridin-4- | 292.353 |
| ylmethyl)benzenesulfonamide | |
| SMR000068045 | 382.475 |
| 3-(tert-butyl)-N-[(6-fluoro-4H-1,3- | 347.383 |
| benzodioxin-8-yl)methyl]-1-methyl-1H- | |
| pyrazole-5-carboxamide | |
| MLS001124721 | 391.509 |
| 3,3′-Diethylthiazolinocarbocyanine iodide | 396.353 |
| MLS001240923 | 499.941 |
| T5337170 | 382.409 |
| BAS 07204618 | 293.359 |
| Carboxyamidotriazole | 424.668 |
| ST50941838 | 405.297 |
| MLS000687652 | 447.504 |
| MLS000732313 | 365.425 |
| T5403634 | 449.52 |
| BAS 05598377 | 290.38 |
| Ambcb81049924 | 377.482 |
| MLS002702480 | 465.488 |
| MLS002694437 | 408.496 |
| 4-(3,4-Dihydro-1H-isoquinolin-2-yl)-8- | 318.347 |
| fluoro-5H-pyrimido[5,4-b]indole | |
| SMR000241542 | 455.341 |
| ST094020 | 326.798 |
| SMR000211540 | 413.35 |
| MLS000705900 | 376.289 |
| Ambcb42757923 | 368.379 |
| MLS003315670 | 521.569 |
| T0501-4035 | 440.305 |
| MLS002702133 | 560.589 |
| 4-[4-(4-methoxyphenyl)piperazino]-5H- | 359.424 |
| pyrimido[5,4-b]indole | |
| MLS002632722 | 483.607 |
| MLS002302684 | 434.502 |
| 2,4,6-trimethyl-N-(pyridin-4- | 290.38 |
| ylmethyl)benzenesulfonamide | |
| T6023590 | 431.549 |
| F0558-0175 | 524.654 |
| T5234163 | 315.39 |
| MLS001122505 | 363.456 |
| CAS-66-81-9 | 281.347 |
| MLS001101361 | 339.366 |
| MLS001144057 | 318.365 |
| Ambcb90456311 | 377.482 |
| Boc-KS | 693.917 |
| (+)-Emetine dihydrochloride hydrate | 535.115 |
| 4-(2-methylimidazo[1,2-a]pyridin-3-yl)-N- | 320.411 |
| (3-methylphenyl)-1,3-thiazol-2-amine | |
| MLS003120811 | 472.581 |
| MLS001018548 | 261.385 |
| 1-[1,1′-Biphenyl]-4-yl-2-(4-imino-1(4H)- | 369.255 |
| pyridinyl)ethanone | |
| GNF-Pf-4659 | 319.848 |
| 5,6-dimethyl-4-{4-[2-(4- | 382.522 |
| methylphenoxy)ethyl]piperazin-1- | |
| yl}thieno[2,3-d]pyrimidine | |
| ST50775950 | 368.449 |
| AGN-PC-00MQWB | 390.315 |
| MLS000734694 | 358.432 |
| MLS000692856 | 309.405 |
| T6090485 | 509.643 |
| ZINC04354521 | 351.428 |
| T5626573 | 393.778 |
| SMR000044829 | 385.844 |
| MLS003107990 | 341.402 |
| SMR000007522 | 433.567 |
| ChemDiv1_019321 | 511.636 |
| ZINC04967005 | 408.556 |
| MLS000712179 | 436.367 |
| T0507-0244 | 307.412 |
| SMR000718391 | 502.674 |
| T0512-9975 | 360.387 |
| AC1LD72C | 343.423 |
| 4-bromo-2,5-dimethyl-N-(pyridin-4- | 355.25 |
| ylmethyl)benzenesulfonamide | |
| MLS000717833 | 390.297 |
| MLS001030349 | 574.668 |
| T6120097 | 393.414 |
| ASN 04448329 | 432.554 |
| ST50323391 | 271.744 |
| 2-methyl-3,5-bis(4- | 363.792 |
| methylphenyl)isoxazol-2-ium | |
| MLS003120629 | 388.415 |
| MLS000553012 | 474.4 |
| SMR000625125 | 496.624 |
| T0508-0735 | 356.442 |
| Ambcb40308772 | 395.451 |
| MLS001032885 | 502.41 |
| SMR000036350 | 335.27 |
| GNF-Pf-1678 | 366.844 |
| MLS000040860 | 302.346 |
| T5565081 | 464.535 |
| SMR000354849 | 507.863 |
| Verrucarin A 9,10-epoxide | 518.552 |
| SMR000274842 | 480.597 |
| SMR000241871 | 447.594 |
| SMR000623161 | 482.615 |
| 5423-98-3 | 327.4 |
| MLS001159633 | 502.482 |
| MLS002473459 | 323.412 |
| MLS000733096 | 413.428 |
| TCMDC-125620 | 339.313 |
| N1-(2,4-difluorophenyl)-4-[5- | 416.411 |
| (trifluoromethyl)-2-pyridyl]-1,4- | |
| diazepane-1-carbothioamide | |
| MLS003116118 | 479.529 |
| MLS003678910 | 353.416 |
| T6069554 | 351.355 |

### EXAMPLES

### Materials & Methods

### Cell Lines

U937 cells were purchased from ATCC and grown in RPMI supplemented with 10% FBS. 32Dc13 cells were purchased from ATCC and grown in RPMI with lng/mL of rmIL-3. The 293GPG retroviral packaging cell line (a gift of Richard Mulligan, Harvard University) was grown in DMEM medium supplemented with 10% FBS, tetracycline (1 mg/mL), G418 (0.3 mg/mL) and puromycin (2 mg/mL).

### Generation of Retroviruses

NR2F6 cDNA (a kind gift from John Ladias, Harvard University) was subcloned into the pcDNA3.1V5/HIS vector (Invitrogen). V5-tagged NR2F6 was subsequently subcloned into the MMP retrovector such that it lay upstream of an IRES (internal ribosome entry sequence)-GFP cassette. VSV-G pseudotyped retroviral particles were generated by transient transfection of 293GPG cells with 25 ug of plasmid in lipofectamine 2000. Viral supernatant was collected for seven days from cultures of these cells in media containing high glucose DMEM with 10% FBS that contained no tetracycline, G418 or puromycin. Viral stocks were concentrated by centrifugation at 16,500 RPM for 90 minutes. In some experiments producer cell lines that stably express the MMP-NR2F6 or MMP-GFP retroviral construct were generated for the production of viral stock. Virus was produced from these cell lines by culturing in high glucose DMEM that contained no tetracycline, G418 or puromycin. Following 7 days of culture viral stock was concentrated by centrifugation at 16,500 RPM for 90 minutes. For U937 and 32D infections, cells were infected at a multiplicity of infection (MOI) of 2. GFP positive cells were harvested by FACS 48 h after infection.

### Patient Material

Leukemia and healthy BM cells, collected with informed consent and with institutional ethics board approval and stored in our tissue bank, were used to assess expression of NR2F6. The French-American British classification of the AML samples consisted of 6 AML-M4, 7 AML-M4eo, 1 AML-M3 and 1 AML-M1.

### Real-Time PCR

RNA was isolated from 1×10⁶ cells using Trizol reagent (Invitrogen) and first strand cDNA was synthesized using SuperScript reverse transcriptase (Qiagen) according to manufacturer's instructions. Real time PCR was performed according to manufacturer's instructions using SYBR Green Master Mix (Applied Biosystems, Foster City, Calif.) and analysed using the delta-delta CT method. The forward and reverse primers used for NR2F6 are 5′-TCTCCCAGCTGTTCTTCATGC-3′ (SEQ ID NO:18) and 5′-CCAGTTGAAGGTACTCCCCG-3′ (SEQ ID NO:19), respectively, and for GAPDH 5′-GGCCTCCAAGGAGTAAGACC-3′ (SEQ ID NO: 20) and 5′-AGGGGTCTACATGGCAACTG-3′ (SEQ ID NO: 21). Threshold cycle (C_{T}) values were calculated in each sample for NR2F6 and normalized to the C_{T} for the housekeeping gene GAPDH (delta-C_{T}). The relative quantity of NR2F6 expression in samples relative to control was be determined as the delta-C_{T} of the sample subtracted from the delta-C_{T} of control, to the exponent 2(delta-delta-C_{T}). For analysis of NR2F6 expression in patient samples the mean delta-C_{T} of all normal samples was used to calculate delta-delta-C_{T} values.

### Differentiation Assessment and Induction

Differentiation was induced in the U937 cell line by treatment with 10 nM TPA (Sigma), 1 uM ATRA (Sigma), or 1.25% v/v DMSO (Sigma) respectively. Immunostaining for the maturation marker CD11b (eBioscience) was performed for twenty minutes in the dark according to manufacturer's instructions and cells were analysed by flow cytometry. Nitroblue tetrazolium (NBT) reduction test (Sigma) was performed according to the manufacturer's instructions, with a minimum of 300 cells scored per slide in three different fields of view. Each experimental timepoint was conducted in triplicate.

### Bone Marrow Transduction

Using the retroviral constructs described above, expression of NR2F6 was forced in primary murine BM cells and monitor the effects on differentiation using colony assays. Donor 12-week old C57B1/6 mice were given 5 fluorouracil, 150 μg/g body mass, by intraperitoneal injection and humanely killed ninety-six hours later. Bone marrow was collected from femurs and tibiae and cultured in Iscove's Modified Dulbecco's Medium supplemented with foetal bovine serum (5%), c-Kit ligand conditioned medium (3%), Flt-3 (30 ng/mL), and TPO (30 ng/mL), conditions that minimize differentiation but initiate cycling of long-term repopulating cells. After 24 hours of culture, the cells were infected with MMP-GFP or MMP-NR2F6 retroviral supernatant at a multiplicity of infection (MOI) of 100. Forty-eight hours after retroviral infection GFP-positive cells were collected by fluorescence activated cell sorting (FACS).

### Methylcellulose Colonies

Following bone marrow transduction with MMP-GFP or MMP-NR2F6 GFP positive cells were collected by FACS and plated in methylcellulose medium supplemented with cytokines (c-Kit ligand, IL-3, IL-6, and erythropoietin) that favour multi-lineage terminal differentiation (Methocult GF 3434, Stem Cell Technologies). Colony formation was evaluated after 12-14 days; clusters containing more than 30 cells will be scored as a colony. Accuracy of colony identification and morphological maturity of colony cells was confirmed by spreading and staining individual colonies on glass slides. Cultures were evaluated for their number of colonies, colony lineage (granulocyte-monocyte, erythroid, or mixed) and morphology. GFP expression was confirmed by fluorescence microscopy. Differences in colony numbers between NR2F6 and controls will be tested for statistical significance with Student's t-test. Secondary colony formation was tested by harvesting an entire primary colony cultures, washing the cells two times with PBS, and plating 10,000 cells in methylcellulose a second time. Secondary colonies were enumerated 12-14 days following a secondary plating.

### Ex Vivo Suspension Culture

Following transduction of mouse bone marrow with MMP-GFP or MMP-NR2F6, cells were placed unsorted into cultured in IMDM with 5% FBS, 10% v/v IL-3 conditioned medium from WEHI cells, 1 ng/mL IL-6 and 3% v/v c-kit ligand conditioned medium. Following ten days of culture the cells were washed twice with PBS, stained with either fluorescently labelled c-kit or with fluorescently labelled CD11b and GR-1, and analysed by flow cytometry.

### Hematopoietic Stem Cell Transplants

Bone marrow transplant recipients were generated that received either chimerical NR2F6 or GFP transduced grafts or grafts that contained 100% sorted bone marrow cells.

To generate recipients transplanted with bone marrow grafts containing a chimera of transduced and wild-type cells 5FU-primed C57B1/6 bone marrow cells were transduced with either MMP-GFP or MMP-NR2F6 as described above. Cells were then sorted by FACS. Transduced (GFP or NR2F6) and untransduced donor cells were mixed at a ratio of between 10:90 to 30:70 (transduced:untransduced), maintaining a constant total graft size of between 4×10⁴ to 1×10⁵ cells per recipient. All recipients of a given cohort received the same graft size. Primary chimerical transplants were performed as described. In some experiments chimerical transplant recipients were harvest at 4-6 weeks post transplant for analysis, and bone marrow was transplanted into another lethally irradiated mouse by tail-vein injection. Secondary recipients of chimerical bone marrow were harvested at either early time points 4-6 weeks or at late time points 12-16 weeks.

To generate recipients transplanted with bone marrow grafts containing 100% transduced bone marrow cells 5FU-primed C57B1/6 bone marrow cells were transduced with either MMP-GFP or MMP-NR2F6 as described above. Cells were then sorted by FACS and introduced into recipient mice by tail vein injection at a dosage of between 4×10⁴ and 1×10⁵ cells per recipient. All recipients of a given cohort received the same graft size. Recipient C57B1/6 mice were treated with 900 cGy prior to transplantation—it was previously determined that this radiation dose is the lowest reliably lethal dose for this strain.

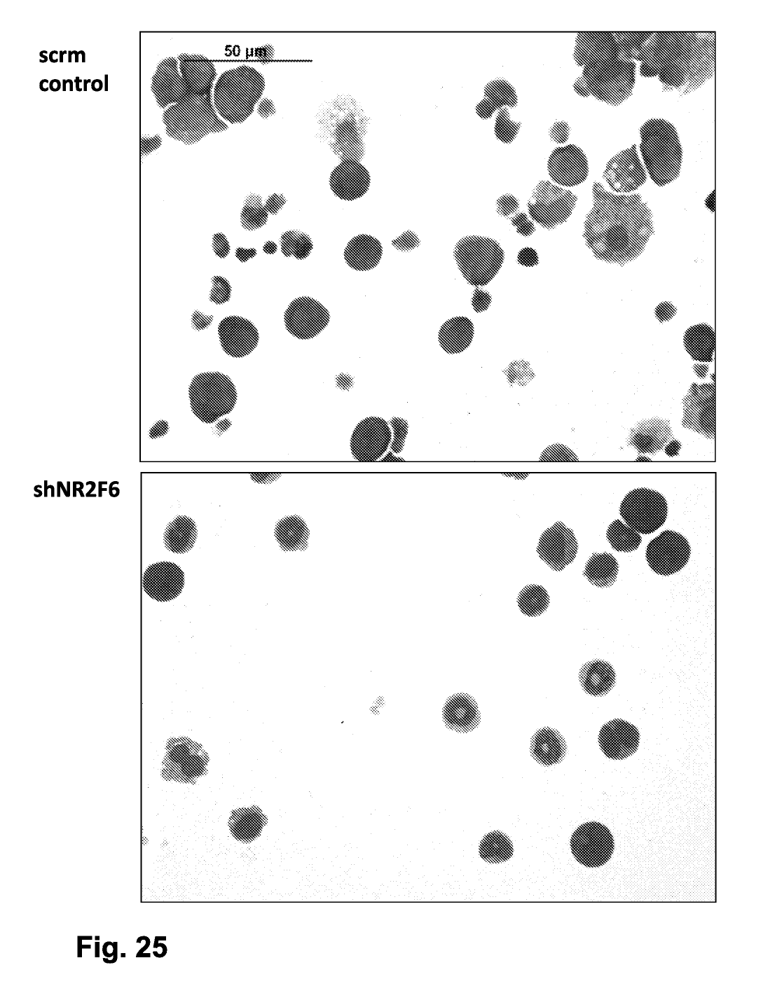

For the competitive transplant experiment (FIG. 25) animals were prepared as described in the generation of recipients transplanted with bone marrow grafts containing a chimera of transduced and wild-type cells. The percentage of marked cells was determined based on expression of GFP using flow cytometry.

### Histological Sections and Cytospins

Immediately following sacrifice of animals tissues were rinsed in PBS and fixed for 24 hours in buffered formalin before being given off to the Sunnybrook Research Institute Histology facility for paraffin embedding, slicing and staining with hematoxylin and eosin. Bone tissues were decalcified following fixation before further processing. Cytospins were prepared by centrifuging single celled suspensions onto glass slides using a Shandon cytocentrifuge. Cytospins were air dried, and fixed in methanol before staining with May-Gruwald and Giemsa stains. Cytospins were coverslipped following treatment with a toluene-based synthetic resin mounting medium.

### Peripheral Blood Counts:

Bone marrow transplant recipients that received grafts containing 100% transduced bone marrow cells were bleed at 4 weeks post-transplant from the Saphenous vein. Alternatively, moribund animals were bled by cardiac puncture just prior to death. To give matched data, a GFP control animal was analysed with every NR2F6 moribund animal analysed. Blood was collected using a heparinized capillary tube and taken to the Toronto Centre for Phenogenomics for acquisition of haematological parameters on a Hemavet analyser.

### Analysis of Hematopoietic Stem Cell Subsets:

Bone marrow transplant recipients that received grafts containing 100% transduced bone marrow cells were humanely sacrificed at four weeks post-transplant. Red blood cells were lysed and bone marrow washed two times with PBS. Bone marrow cells were then stained with biotin CD3, biotin CD45R/B220 (RA3-6B2), biotin CD11b (M1/70), biotin erythroid marker (TER-119), biotin Ly-6G (RB6-8C5), c-kit APC, sca-1 PE-Cy7 and either CD34 PE or CD49b PE (all eBioscience) in the dark. Bone marrow was washed once and incubated with streptavidin PE-Cy5 for 20 minutes in the dark. Bone marrow was washed twice and analysed using flow cytometry on a Becton Dickinson LSR II. All samples analysed were gated based on FSC/SSC and GFP+ cells.

The population of lineage⁻ Sca-1⁺ c-kit⁺ (LSK) is highly enriched for hematopoietic stem cell activity. This population was analysed and further subdivided based on the expression of the CD34 and CD49b antigen. Whereas the CD34^{-/low} and the CD49b^{-/low} population of LSK cells are enriched for long-term hematopoietic stem cells, the CD34⁺ and CD49b+ population of LSK cells are composed of short term hematopoietic stem cells.

Overexpression of NR2F6 in bone marrow cells transplanted into a mouse with chimeric bone marrow results in transplanted hematopoietic cells possessing reduced ability to generate T cells

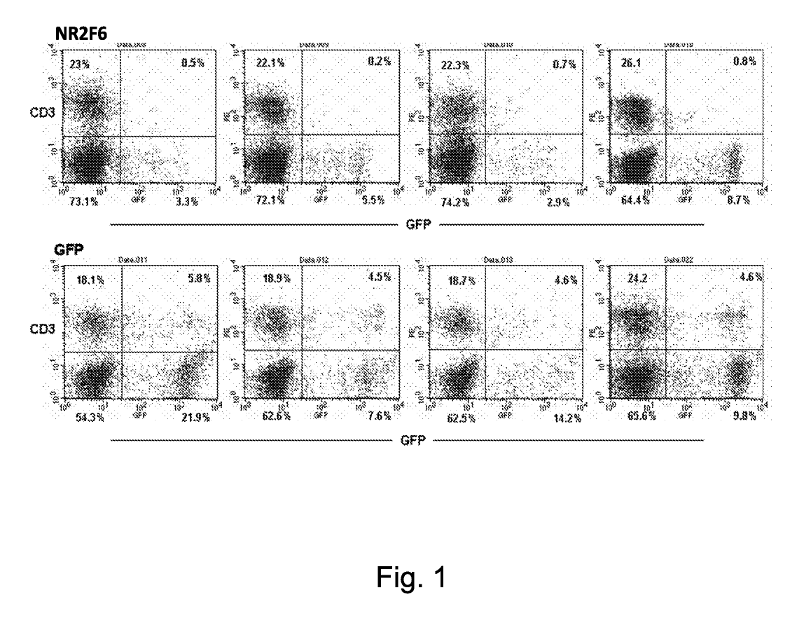

FIG. 1 shows that bone marrow cells that over-express NR2F6 do not contribute to peripheral blood T-cells in a chimerical bone marrow transplant model.

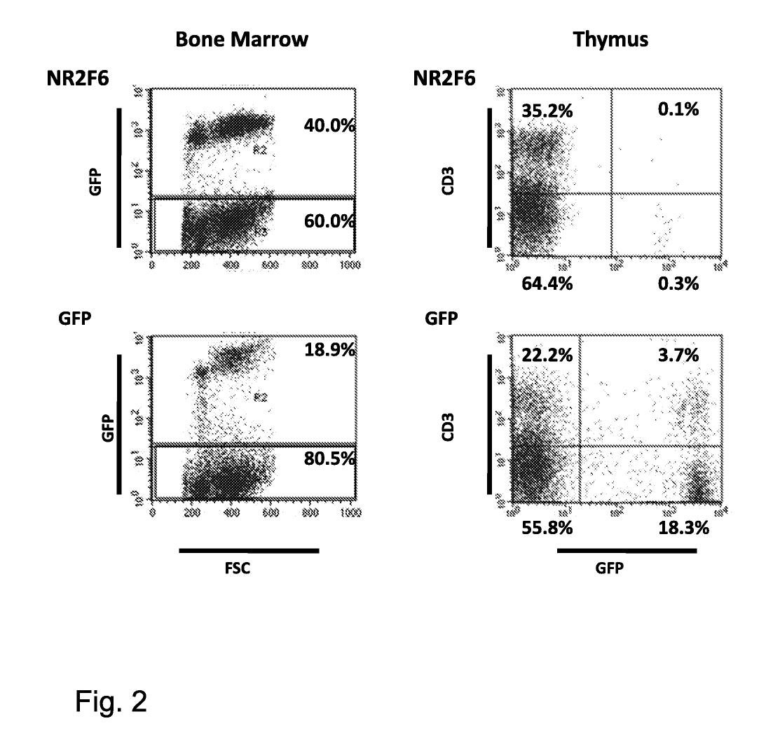

NR2F6 over-expressing bone marrow is at a competitive disadvantage to normal bone marrow with respect to survival and proliferation in the thymus of chimerical bone marrow transplant recipients, shown here in representative animals. FIG. 2.

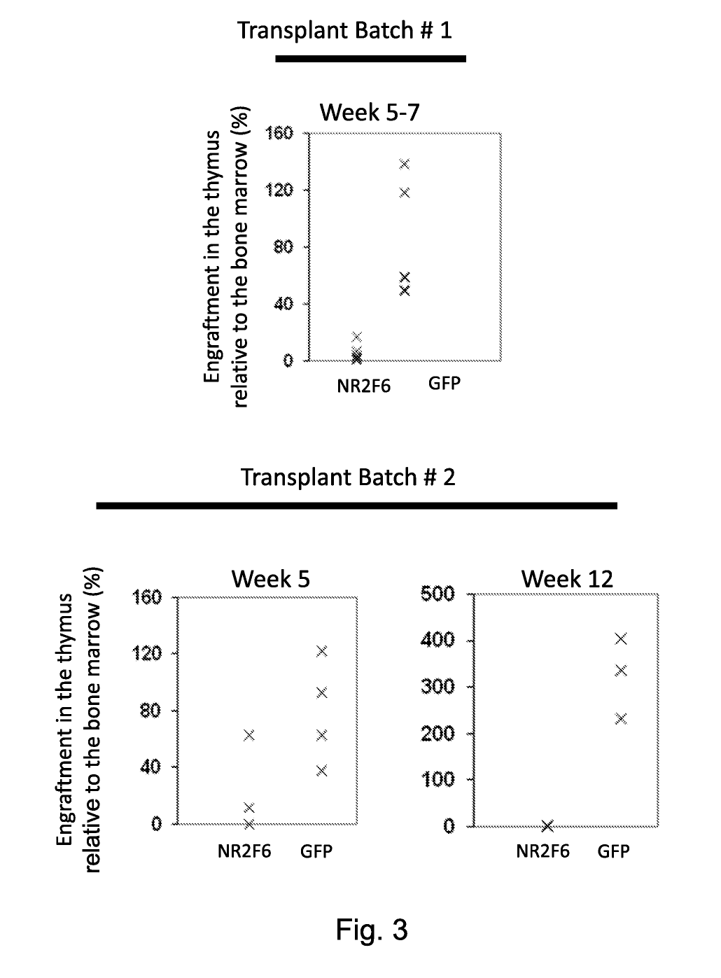

NR2F6 over-expressing bone marrow is at a competitive disadvantage to normal bone marrow with respect to survival and proliferation in the thymus of chimerical bone marrow transplant recipients, shown here as a summary of two independent experiments. FIG. 3

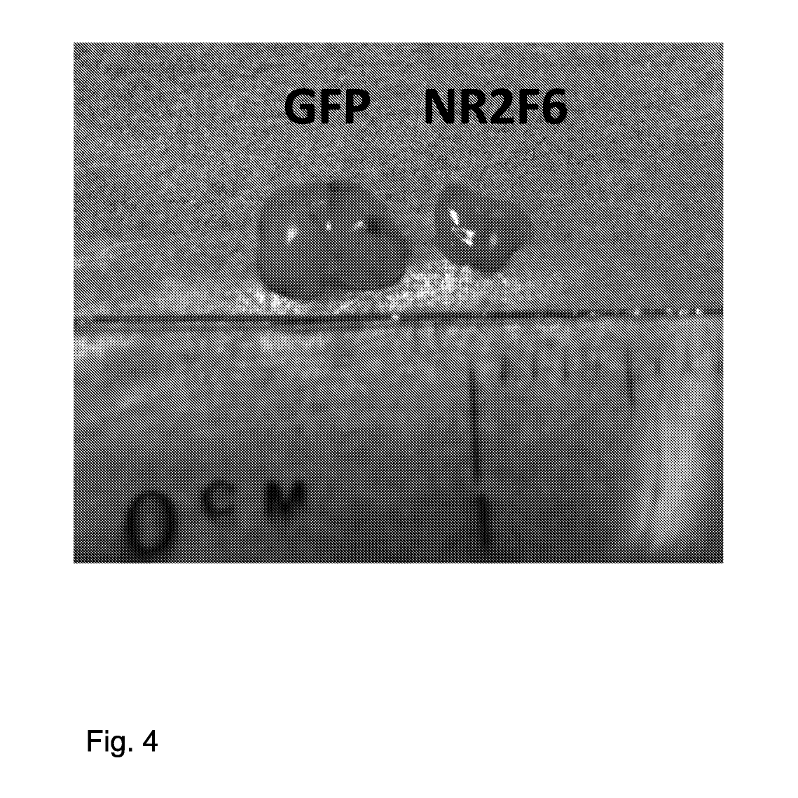

Reduction in the size of the thymus in animals that had been transplanted with bone marrow that over-expresses NR2F6 in contrast to control bone marrow transplant recipients. FIG. 4

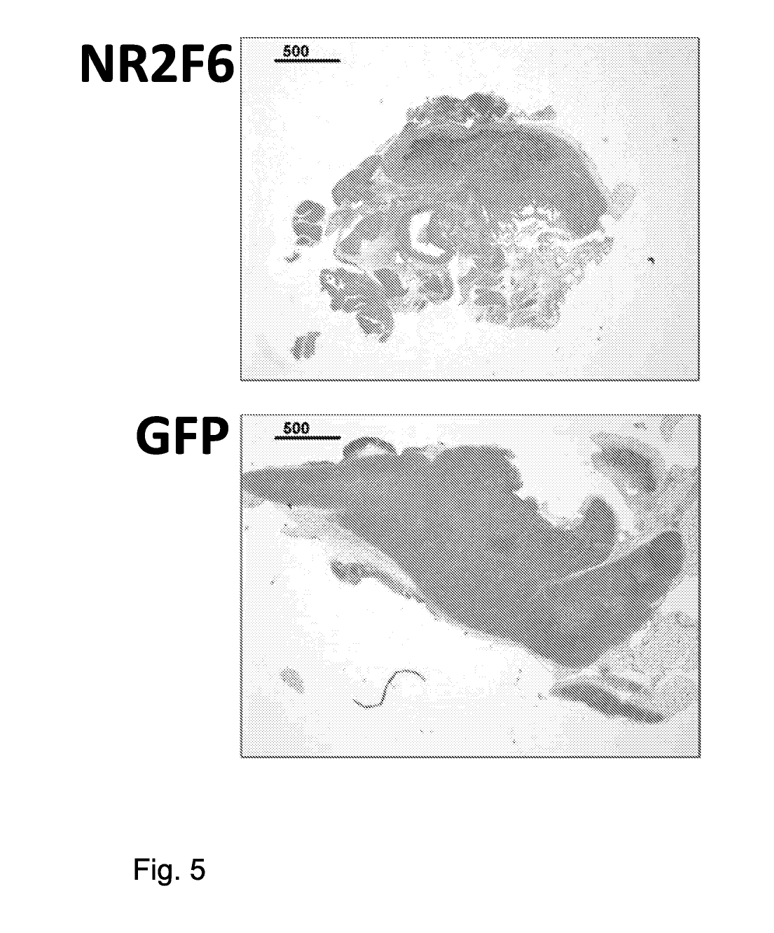

Reduction in the cellularity of the thymic cortex in bone marrow transplant recipients that received bone marrow that over-expressed NR2F6. FIG. 5

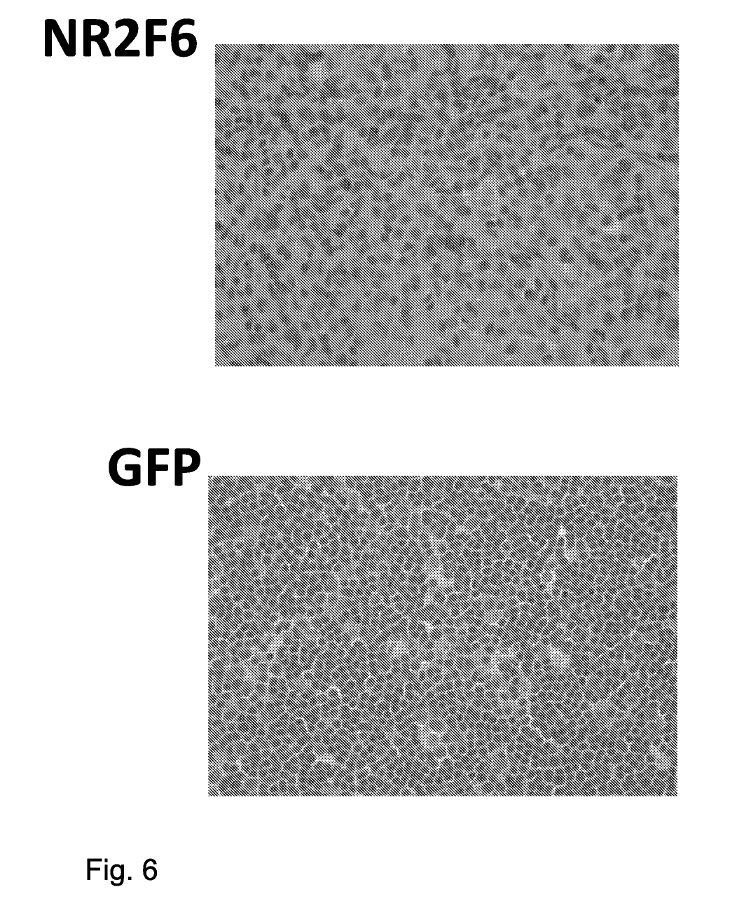

Reduction in the cellularity of the thymic cortex in bone marrow transplant recipients that received bone marrow that over-expressed NR2F6. FIG. 6

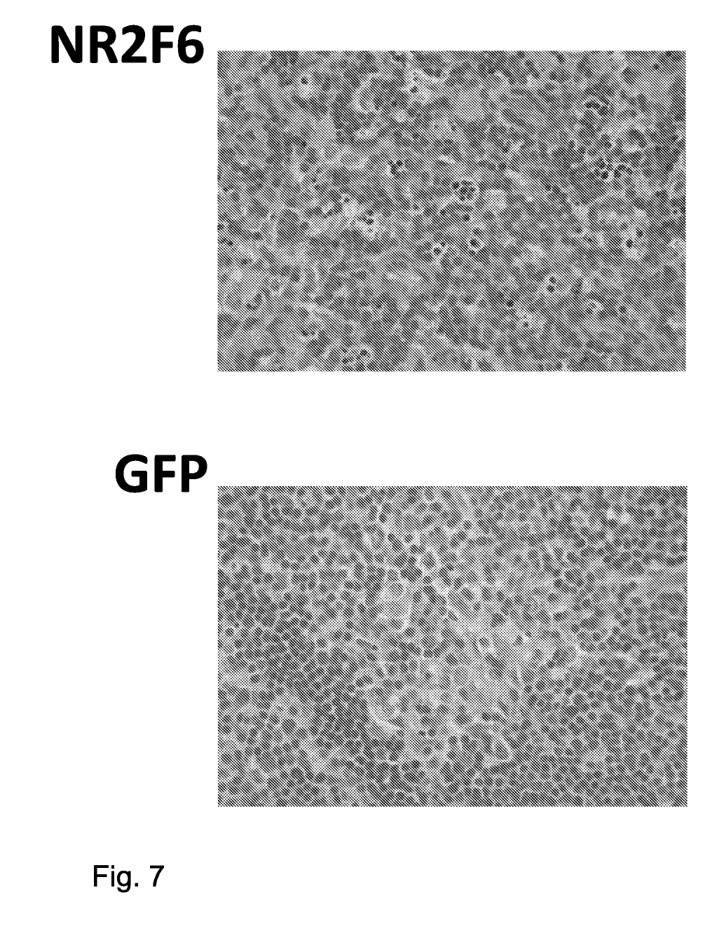

Cell death in the thymic medulla in bone marrow transplant recipients that received bone marrow that over-expressed NR2F6. FIG. 7

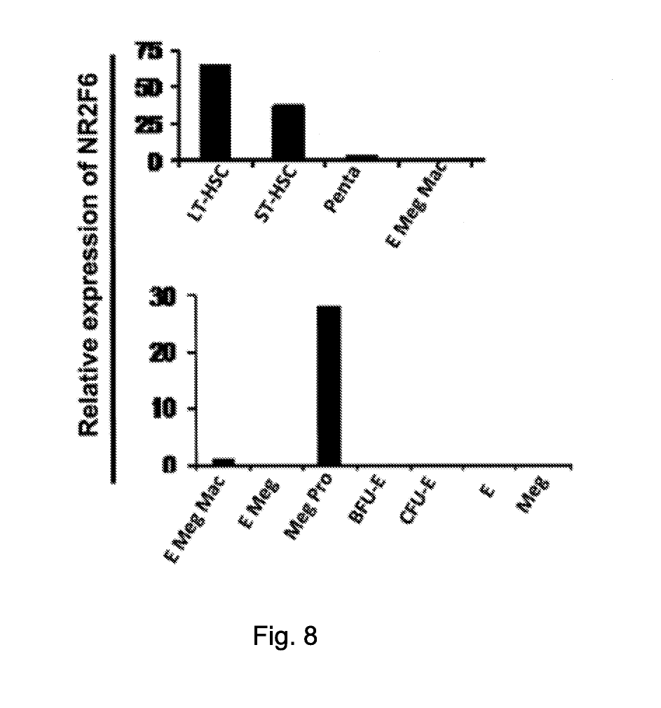

NR2F6 is highly expressed in both long and short term haematopoietic stem cells and that expression of NR2F6 in bone marrow hierarchy is differentially expressed. FIG. 8.

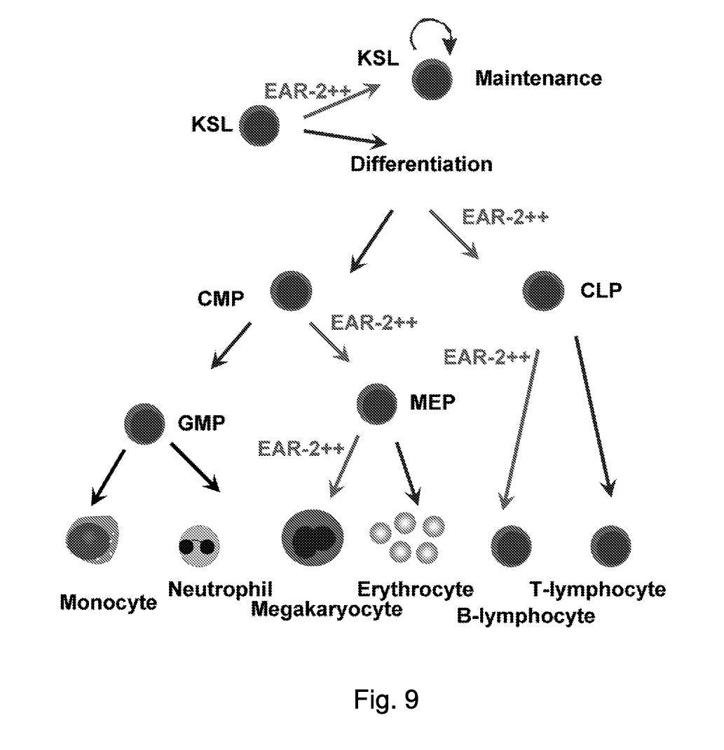

A model whereby NR2F6 is a gatekeeper of lineage selection. FIG. 9.

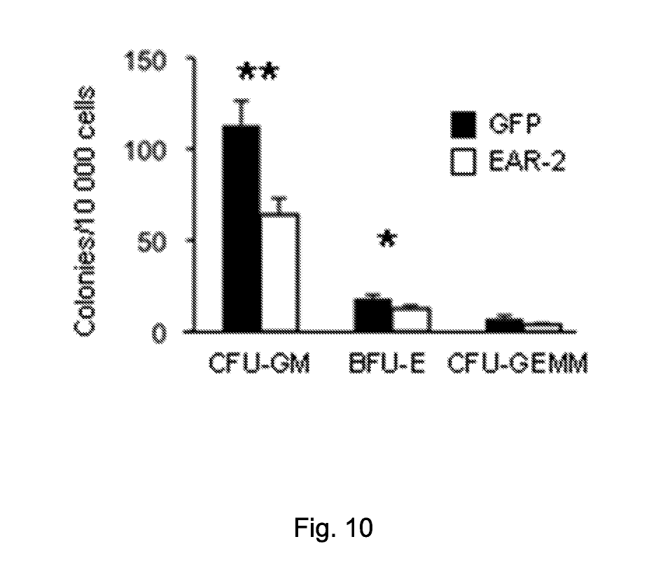

Over expression of NR2F6 in bone marrow cells inhibits progenitor cell colony formation in methylcellulose along the myeloid and erythroid lineages. FIG. 10.

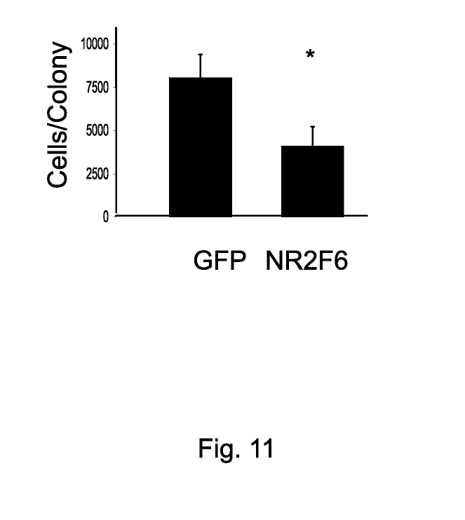

Over expression of NR2F6 in bone marrow cells inhibits differentiation of bone marrow cells evidenced by generation of smaller colonies in NR2F6 transduced cultures. FIG. 11.

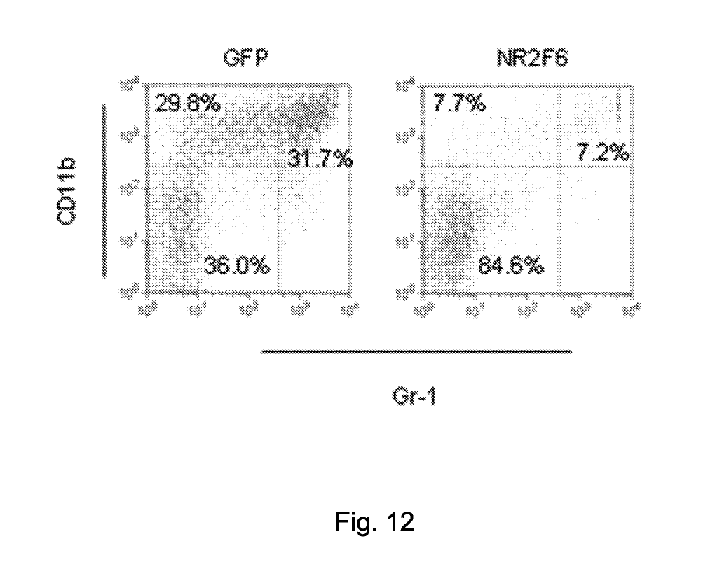

Over expression of NR2F6 in bone marrow cells inhibits differentiation of progenitor cells in suspension culture in to granulocytes. Granulocytes were assessed in cultures by flow cytometry for expression of the surface antigens CD11b and Gr-1. FIG. 12.

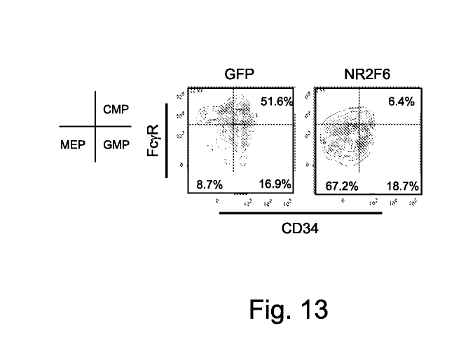

Modulating the expression of NR2F6 in a chimerical bone marrow mouse model (30% NR2F6 transduced, 70% untransduced cells) causes a block in the ability of hematopoietic stem cells to differentiate into hematopoietic progenitor cells, Specifically, the megakaryocyte-erythroid progenitor cell (MEP) while decreasing the size of the common myeloid progenitor cell population as well as the granulocyte-monocyte progenitor (GMP) cell population. FIG. 13.

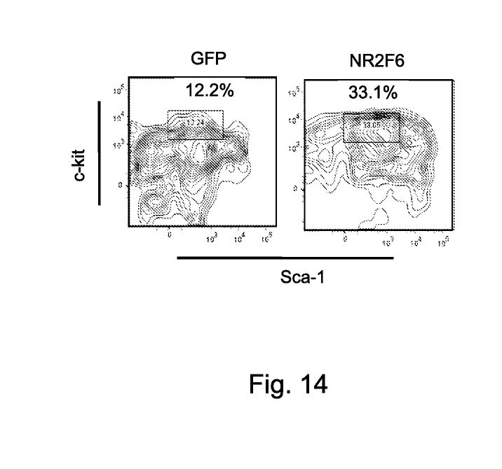

Modulating the expression of NR2F6 in a chimerical bone marrow mouse model (30% NR2F6 transduced, 70% untransduced cells) increases hematopoietic stem cell differentiation into the common lymphoid progenitor cells. FIG. 14.

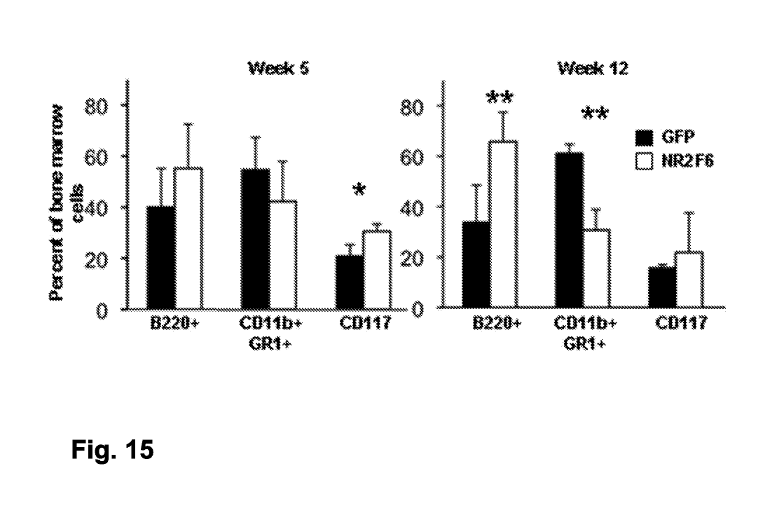

Mice that over expression of NR2F6 in their bone marrow cells have skewed production of immature blood cells in the bone marrow. At 5 weeks post transplantation we observed more c-kit±cells in the bone marrow, indicative of an increase in immature cells, While at 12 weeks post transplant we observed increased production of B220+ cells (immature B-cells) and decreased production of CD11b+/GR1+ cells (granulocytes). FIG. 15.

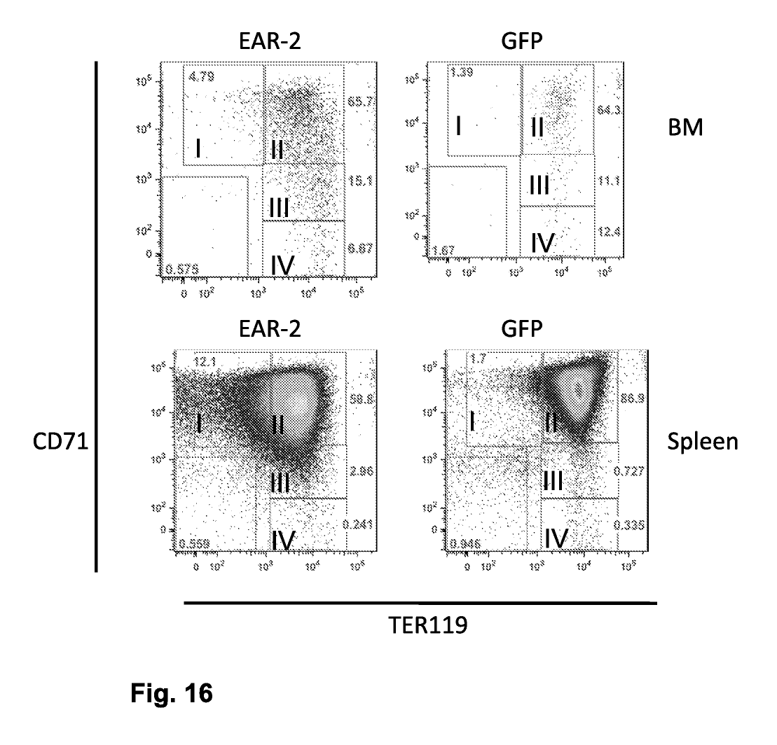

Mice that over expression of NR2F6 in their bone marrow cells have a block at the pro-erythroblast stage of blood cell development both in the bone marrow and in the spleen of animals that have excessive expression of NR2F6. FIG. 16.

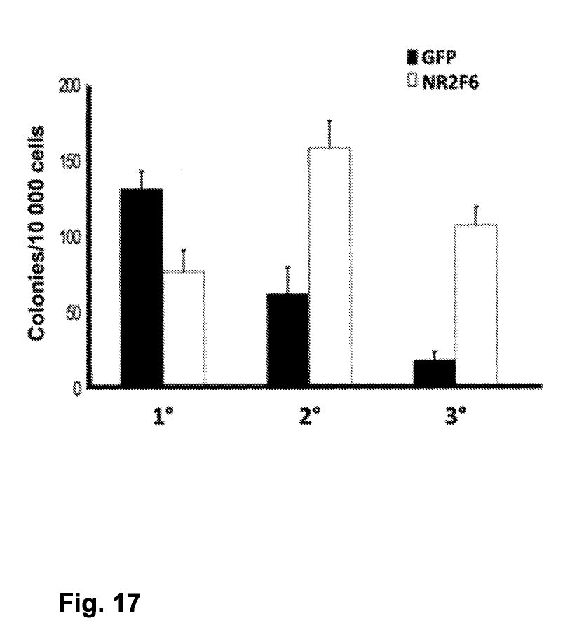

Serial replating of bone marrow from mice that over express NR2F6 in their bone marrow. Bone marrow was grown in methylcellulose medium containing cytokines that promoted multi lineage differentiation. We observe a decrease in the ability of the cells to differentiate, evidenced by fewer primary colonies, but observed an increase in the self-renewal of the bone marrow cells evidenced by ability to serially replate. FIG. 17.

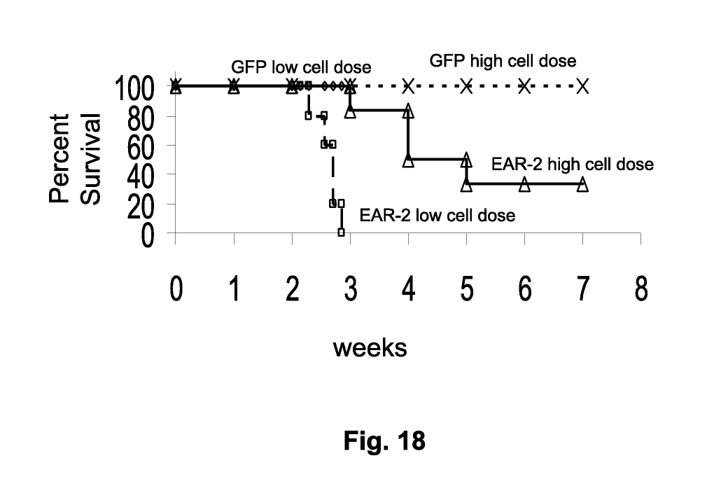

NR2F6 impairs the formation of mature red blood cells in animals that over-express NR2F6 in their bone marrow. This is demonstrated by a reduction in radioprotection, survival following a dose of lethal radiation, in animals that received bone marrow that over-expressed NR2F6. FIG. 18.

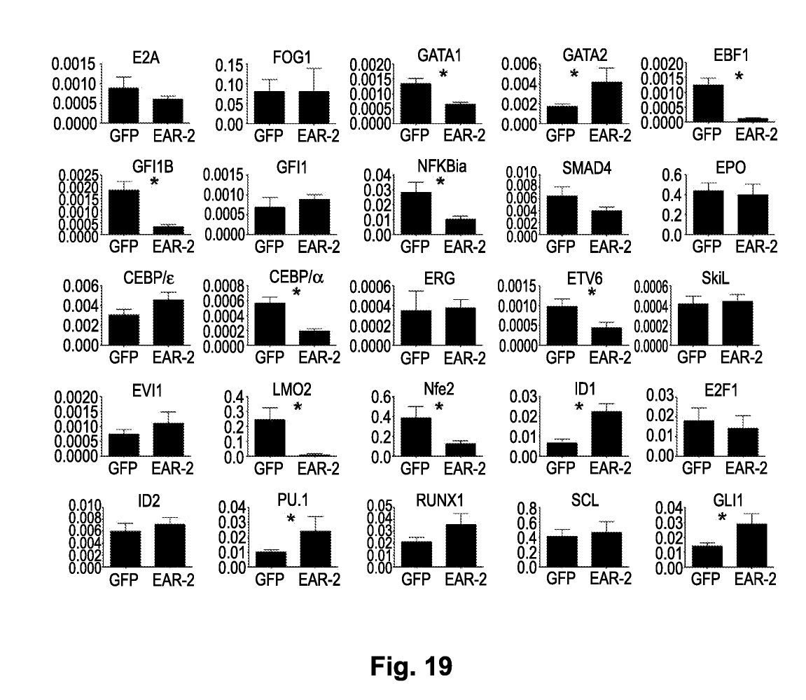

Aberrant gene expression in key genes that govern erythropoiesis in the pro-erythroblast population of animals that over-express NR2F6 and vector control animals. FIG. 19.

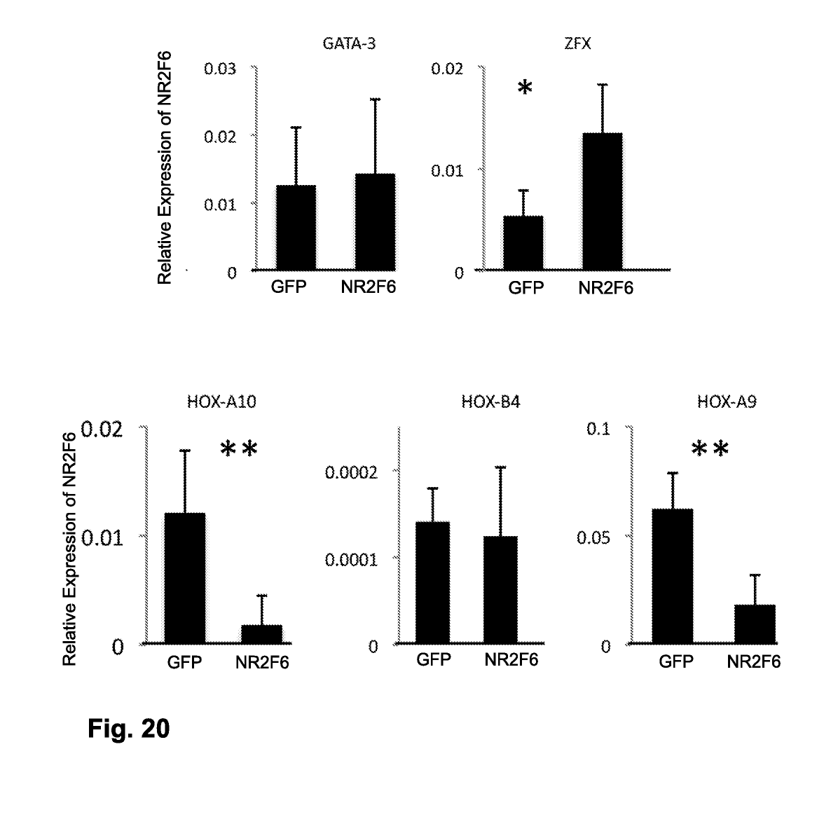

Aberrant gene expression in key genes that govern hema opoiesis in the KSL population of bone marrow stem and progenitor cells in animals that over-express NR2F6 and vector control animals. FIG. 20.

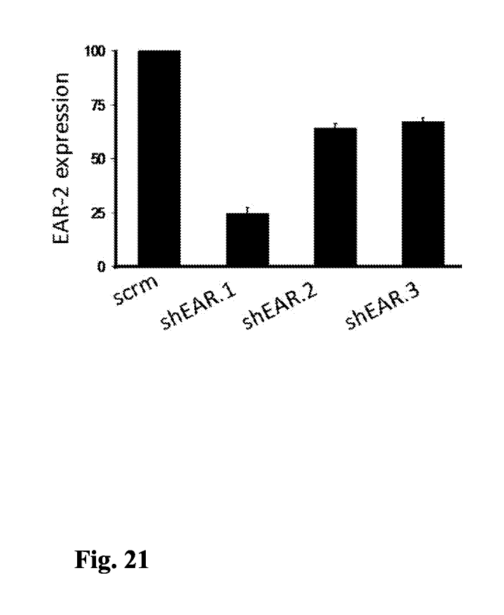

Quantification of NR2F6 (EAR-2) protein levels, determined by immunoblot and quantified using densitometry, in human 32Dcl3 undifferentiated hematopoietic cells that were treated with NR2F6 shRNA or a hairpin control. FIG. 21.

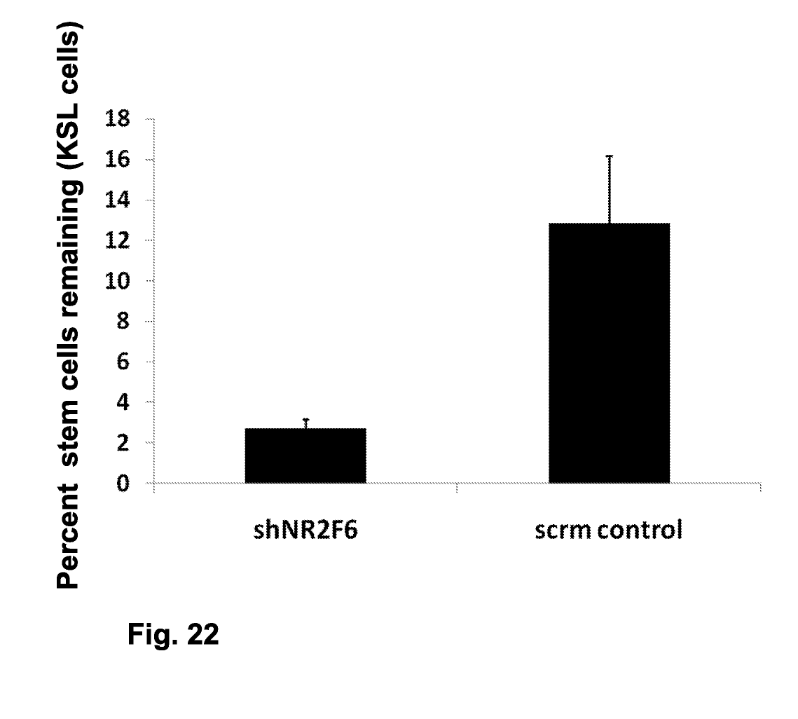

shRNA to NR2F6 rapidly induces differentiation of bone marrow stem cells ex vivo. Here we show a reduction in the number of KSL bone marrow cells remaining in ex vivo cultures. FIG. 22.

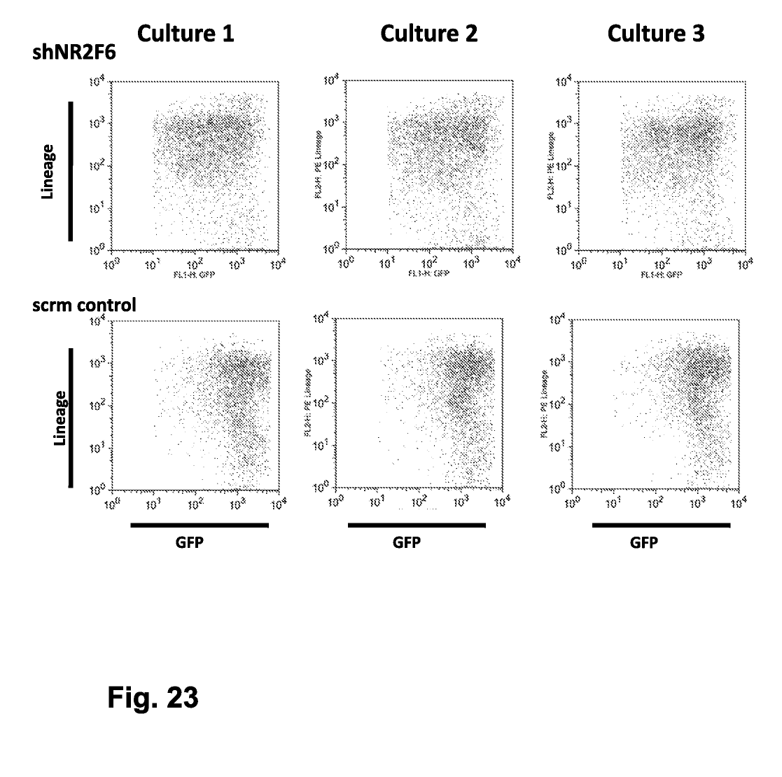

shRNA to NR2F6 rapidly induces differentiation of bone marrow stem cells ex vivo. Here we show a reduction in the number of immature bone marrow cells that are devoid of lineage antigens. FIG. 23.

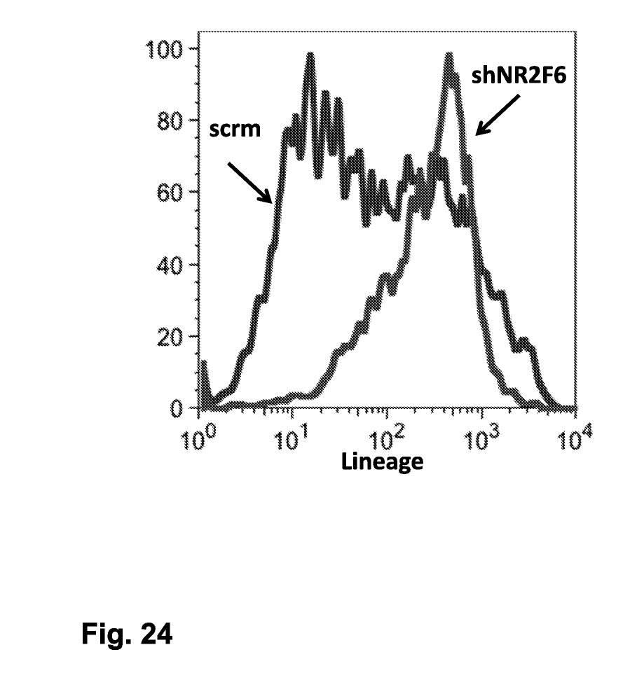

shRNA to NR2F6 rapidly induces differentiation of bone marrow stem cells ex vivo. Here we show a reduction in the number of immature bone marrow cells that are devoid of lineage antigens. FIG. 24.

shRNA to NR2F6 rapidly induces differentiation of bone marrow stem cells ex vivo. Here we show a cytospin preparation that shows morphologically a reduction in the number of immature bone marrow cells. Samples that were treated with NR2F6 shRNA rapidly differentiated in to granulocytic cells. FIG. 25.

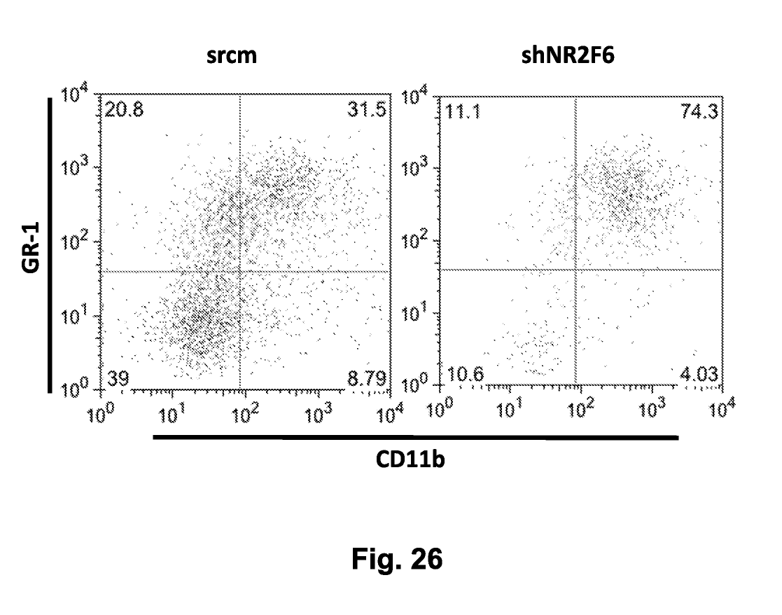

shRNA to NR2F6 rapidly induces differentiation of bone marrow stem cells ex vivo. Here we show flow cytometry dot plots that shows that samples that were treated with NR2F6 shRNA rapidly differentiated in to granulocytic cells. FIG. 26.

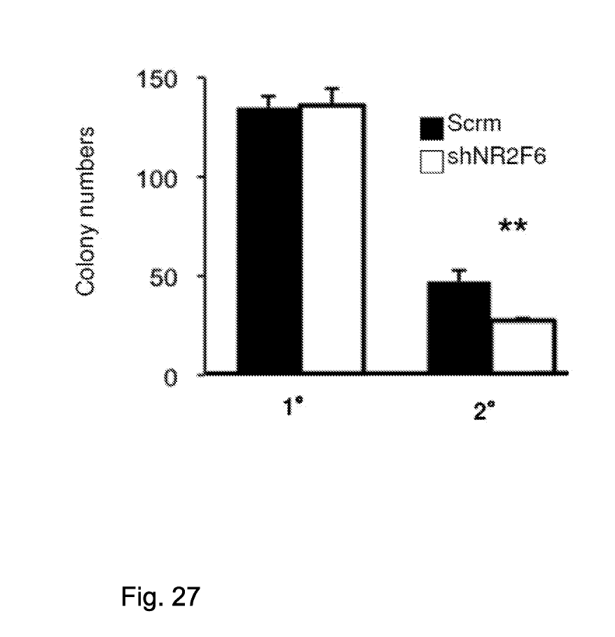

shRNA to NR2F6 rapidly induces differentiation of bone marrow stem cells. Here we show that shRNA to NR2F6 inhibits the formation of secondary bone marrow colonies, hence demonstrating a reduction in the self-renewal ability of the cultures due to differentiation. FIG. 27.

### REFERENCES

1. Ichim, C. V. and R. A. Wells, First among equals: The cancer cell hierarchy. Leukemia and Lymphoma (in press), 2006.
2. Bonnet, D. and J. E. Dick, Human acute myeloid leukemia is organized as a hierarchy that originates from a primitive hematopoietic cell. Nat Med, 1997. 3(7): p. 730-7.
3. Singh, S. K., et al., Identification of a cancer stem cell in human brain tumors. Cancer Res, 2003. 63(18): p. 5821-8.
4. Singh, S. K., et al., Identification of human brain tumour initiating cells. Nature, 2004. 432(7015): p. 396-401.
5. Al-Hajj, M., et al., Prospective identification of tumorigenic breast cancer cells. Proc Natl Acad Sci USA, 2003. 100(7): p. 3983-8.
6. Reya, T., et al., A role for Wnt signalling in self-renewal of haematopoietic stem cells. Nature, 2003. 423(6938): p. 409-14.
7. Stier, S., et al., Notch1 activation increases hematopoietic stem cell self-renewal in vivo and favors lymphoid over myeloid lineage outcome. Blood, 2002. 99(7): p. 2369-78.
8. Karlsson, G., et al., Smad4 is critical for self-renewal of hematopoietic stem cells. J Exp Med, 2007. 204(3): p. 467-74.
9. Schnabel, C. A., Y. Jacobs, and M. L. Cleary, HoxA9-mediated immortalization of myeloid progenitors requires functional interactions with TALE cofactors Pbx and Meis. Oncogene, 2000. 19(5): p. 608-16.
10. Thorsteinsdottir, U., et al., Overexpression of the myeloid leukemia-associated Hoxa9 gene in bone marrow cells induces stem cell expansion. Blood, 2002. 99(1): p. 121-9.
11. Thorsteinsdottir, U., et al., Overexpression of HOXA10 in murine hematopoietic cells perturbs both myeloid and lymphoid differentiation and leads to acute myeloid leukemia. Mol Cell Biol, 1997. 17(1): p. 495-505.
12. Sauvageau, G., et al., Overexpression of HOXB4 in hematopoietic cells causes the selective expansion of more primitive populations in vitro and in vivo. Genes Dev, 1995. 9(14): p. 1753-65.
13. Lessard, J. and G. Sauvageau, Bmi-1 determines the proliferative capacity of normal and leukaemic stem cells. Nature, 2003. 423(6937): p. 255-60.
14. Park, I. K., et al., Bmi-1 is required for maintenance of adult self-renewing haematopoietic stem cells. Nature, 2003. 423(6937): p. 302-5.
15. Ernst, P., et al., Definitive hematopoiesis requires the mixed-lineage leukemia gene. Dev Cell, 2004. 6(3): p. 437-43.
16. Heyworth, C., et al., A GATA-2/estrogen receptor chimera functions as a ligand-dependent negative regulator of self-renewal. Genes Dev, 1999. 13(14): p. 1847-60.
17. Zeng, H., et al., Transcription factor Gfi1 regulates self-renewal and engraftment of hematopoietic stem cells. Embo J, 2004. 23(20): p. 4116-25.
18. Steidl, U., et al., Essential role of Jun family transcription factors in PU.1 knockdown-induced leukemic stem cells. Nat Genet, 2006. 38(11): p. 1269-77.
19. Iwasaki, H., et al., Distinctive and indispensable roles of PU.1 in maintenance of hematopoietic stem cells and their differentiation. Blood, 2005. 106(5): p. 1590-600.
20. White, J. R. and K. Weston, Myb is required for self-renewal in a model system of early hematopoiesis. Oncogene, 2000. 19(9): p. 1196-205.
21. Rebel, V. I., et al., Distinct roles for CREB-binding protein and p300 in hematopoietic stem cell self-renewal. Proc Natl Acad Sci USA, 2002. 99(23): p. 14789-94.
22. Wilson, A., et al., c-Myc controls the balance between hematopoietic stem cell self-renewal and differentiation. Genes Dev, 2004. 18(22): p. 2747-63.
23. Galan-Caridad, J. M., et al., Zfx controls the self-renewal of embryonic and hematopoietic stem cells. Cell, 2007. 129(2): p. 345-57.
24. Zon, L. I., Intrinsic and extrinsic control of haematopoietic stem-cell self-renewal. Nature, 2008. 453(7193): p. 306-13.
25. Nerlov, C. and T. Graf, PU.1 induces myeloid lineage commitment in multipotent hematopoietic progenitors. Genes Dev, 1998. 12(15): p. 2403-12.
26. Nerlov, C., et al., GATA-1 interacts with the myeloid PU.1 transcription factor and represses PU.1-dependent transcription. Blood, 2000. 95(8): p. 2543-51.
27. Lapidot, T., et al., A cell initiating human acute myeloid leukaemia after transplantation into SCID mice. Nature, 1994. 367(6464): p. 645-8.
28. Blair, A., D. E. Hogge, and H. J. Sutherland, Most acute myeloid leukemia progenitor cells with long-term proliferative ability in vitro and in vivo have the phenotype CD34(+)/CD71(−)/HLA-DR. Blood, 1998. 92(11): p. 4325-35.
29. de Lima, M., et al., Implications of potential cure in acute myelogenous leukemia: development of subsequent cancer and return to work. Blood, 1997. 90(12): p. 4719-24.

1. A method of accelerating differentiation of a stem cell comprising administration to a subject having a cancerous condition an effective amount of a composition comprising a synthetic oligonucleotide complementary to a nuclear receptor having a mRNA sequence of at least 75% sequence identity to the mRNA sequence of SEQ ID NO: 5 that induces the RNA interference, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that has been selected based on its ability to inhibits the expression of the nuclear receptor NR2F6 by causing degradation of a ribonucleic acid encoding nuclear receptor NR2F6 by activation of RNA interference.

2. A method of claim 1 wherein the synthetic oligonucleotide consists of a short-interfering ribonucleic acid (siRNA) molecule.

3. A method of claim 1 wherein the synthetic oligonucleotide consists of a short-hairpin ribonucleic acid (shRNA) molecule.

4. A method of claim 1 wherein the synthetic oligonucleotide consists of an antisense ribonucleic acid molecule.

5. A method of inhibiting expression of NR2F6 protein in a subject for a therapeutic purpose, comprising the step of: administering to a subject an effective amount of pharmaceutical composition comprising a synthetic oligonucleotide comprising a sense strand and an antisense strand, wherein the sense and antisense strands form a duplex, and wherein the sense RNA strand comprises SEQ ID NO:5, thereby specifically inhibiting the expression of NR2F6.

6. (canceled)

7. (canceled)

8. The method of claim 1 wherein the effective portion of the oligonucleotide consists of SEQ ID NO: 9, 10, 11 or 12.

9. The method of claim 1 wherein the effective portion of the oligonucleotide consists of SEQ ID NO: 16 or 17.

10. The method of claim 1 wherein the effective portion of the oligonucleotide consists of SEQ ID NO: 13, 14 or 15.

11. A composition comprising an oligonucleotide complementary to a nuclear receptor having a mRNA sequence of at least 75% sequence identity to the mRNA sequence of SEQ ID NO: 5, wherein said nucleotide comprises a sense oligonucleotide strand and an antisense oligonucleotide strand, wherein the sense and antisense oligonucleotide strands form a duplex, and wherein the sense oligonucleotide strand comprises a portion of SEQ ID NO:5 that is selected based on its ability to inhibits the expression of the nuclear receptor NR2F6 by causing degradation of a ribonucleic acid encoding nuclear receptor NR2F6.

12. A composition of claim 11 consisting of a short-interfering ribonucleic acid (siRNA) molecule.

13. A composition of claim 11 consisting of a short-hairpin ribonucleic acid (shRNA) molecule.

14. A composition of claim 11 consisting of an antisense ribonucleic acid molecule.

15. The method of claim 1, wherein the synthetic oligonucleotide is chemically modified.

16. The method of claim 5, wherein the synthetic oligonucleotide is chemically modified.

17. The composition of claim 11, wherein the oligonucleotide complementary to a nuclear receptor is chemically modified.
